(19) United States
(12) Patent Application Publication
(10) Pub. No.: US 2017/0275665 A1
(43) Pub. Date: Sep. 28, 2017

(54) DIRECT CRISPR SPACER ACQUISITION FROM RNA BY A REVERSE-TRANSCRIPTASE-CAS1 FUSION PROTEIN

(71) Applicants: Board of Regents, the University of Texas System, Austin, TX (US); The Board of Trustees of the Leland Stanford Junior University, Palo Alto, CA (US); Carnegie Institution of Washington, Washington, DC (US)

(72) Inventors: Sukrit SILAS, Stanford, CA (US); Georg MOHR, Austin, TX (US); Devaki BHAYA, Stanford, CA (US); Alan M. LAMBOWITZ, Austin, TX (US); Andrew FIRE, Stanford, CA (US)

(21) Appl. No.: 15/440,315

(22) Filed: Feb. 23, 2017

Related U.S. Application Data
(60) Provisional application No. 62/299,526, filed on Feb. 24, 2016.

(51) Int. Cl. C12P 19/34; C12Q 1/68; C12N 9/12; C12N 15/11; C12N 9/22

(52) U.S. Cl. CPC C12P 19/34; C12Y 207/07049; C12Y 301/00; C12N 15/11; C12N 9/22; C12N 9/1276; C12Q 1/6806; C12N 2310/20; C12N 2310/3519; C07K 2319/80

(57) ABSTRACT

The present disclosure provides methods and compositions for the integration of a target RNA or DNA into a DNA substrate. Also provided are methods of forming RNA-DNA bonds and enzymes for performing the same.

This application claims the benefit of U.S. Provisional Patent Application No. 62/299,526, filed Feb. 24, 2016, the entirety of which is incorporated herein by reference.

This invention was made with government support under Grant no. R01 GM037949, R01 GM037951 and R01 GM037706 awarded by the National Institutes of Health. The government has certain rights in the invention.

### BACKGROUND OF THE INVENTION

1. Field of the Invention

The present invention relates generally to the field of molecular biology. More particularly, it concerns methods and compositions for the use of the RT-Cas1 fusion protein.

2. Description of Related Art

RNA-guided host defense mechanisms associated with CRISPR arrays exist in most bacteria and archaea (Barrangou et al., 2007; Marraffini and Sontheimer, 2010). Their target specificity derives from a series of spacers, many of which are identical to DNA sequences from phage, transposon, and plasmid mobilome, interspersed within CRISPR arrays (Bolotin et al., 2005; Mojica et al., 2010; Pourcel et al., 2005). Transcripts from these CRISPR arrays are processed into short structured RNAs, which form a complex with CRISPR-associated (Cas) endonucleases and target invasive nucleic acids, thereby conferring immunity (Brouns et al., 2008; van der Oost et al., 2014). CRISPR-Cas systems have been phylogenetically grouped into five types (Makarova et al., 2011; Makarova et al., 2015). Homologs of the Cas1 and Cas2 genes are conserved across diverse CRISPR types (Makarova et al., 2015; Makarova et al., 2006), with direct evidence for a role in the physical integration of new spacers from invasive DNA into CRISPR arrays in a few Type I and II systems (Yosef et al., 2012; Datsenko et al., 2012; Wei et al., 2015; Heler et al., 2015). Spacer acquisition allows the host to adapt to new threats.

The ability of type III systems to target RNA in addition to DNA (Marraffini and Sontheimer, 2008; Hale et al., 2009; Hale et al., 2012; Tamulaitis et al., 2014; Goldberg et al., 2014; Peng et al., 2015; 2015) raises the possibility of natural spacer acquisition from RNA species. Accordingly, there is a need for methods of direct acquisition of RNA spacers which would add to the handful of known mechanisms for the reverse flow of genetic information from RNA into DNA genomes (Baltimore, D., 1970; Temin and Mizutani, 1970; Greider and Blackburn, 1985; Boeke et al., 1985; Zimmerly et al., 1995; Liu et al., 2002).

### SUMMARY OF THE INVENTION

Embodiments of the present disclosure provide methods and compositions for integrating an oligonucleotide into a double-stranded DNA (dsDNA) substrate comprising: (a) obtaining a dsDNA substrate comprising a Cas1 recognition sequence and at least a first polynucleotide; and (b) providing a Cas1 polypeptide, thereby integrating the first polynucleotide into the dsDNA substrate. In certain aspects, providing the Cas1 polypeptide comprises providing the Cas1 polypeptide and a reverse transcriptase polypeptide. In some aspects, the dsDNA substrate is linear or circular. In some aspects, the first polynucleotide comprises single-stranded RNA (ssRNA), double stranded RNA (dsRNA), single-stranded DNA (ssDNA) and/or dsDNA. In particular aspects, the first polynucleotide comprises ssRNA. Accordingly, some aspects provide an RNA-DNA hybrid. In some aspects, the assay is performed in vivo. In other aspects, the assay is performed in vitro.

In some aspects, the polynucleotide (e.g., ssRNA) has a length of about 10-100 nucleotides or any length derivable thereof, such as 20, 30, 40, 50, 60, 70, 80, or 90 nucleotides. In certain aspects, the polynucleotide has a length of about 20-60 nucleotides, such as 20-50 nucleotides. In particular aspects, the polynucleotide is 34, 35, or 36 nucleotides. In some aspects, more than one polynucleotide is integrated. In some aspects, 2, 3, 4, 5, 6, 10, 10², 10³, 10⁴, 10⁵, 10⁶, or 10⁷ polynucleotides are obtained in step (a). In some aspects, the polynucleotides are obtained by fragmenting RNA or DNA. For example, the fragmentation can be performed by physical fragmentation such as sonication or acoustic shearing. In other aspects, the fragmentation may be performed by enzymatic methods such as a nuclease. In some aspects, long RNA fragments are chemically sheared such as by heat and divalent metal cations.

In certain aspects, the method further comprise providing a reverse transcriptase in addition to the Cas1. In some aspects, the reverse transcriptase (RT) and Cas1 are provided separately. In other aspects, RT and Cas1 are provided as a RT-Cas1 fusion protein. In some aspects, the RT-Cas1 fusion protein is provided in an expression vector. In certain aspects, the RT-Cas1 fusion protein is a bacterial RT-Cas1 fusion protein. For example, the RT-Cas1 fusion can be isolated from cyanobacterium, Arthrospira platensis or the gammaproteobacterium Marinomonas mediterranea. In some aspects, the RT-Cas1 fusion protein comprises an amino acid sequence at least 80% identical to SEQ ID NO: 3. In certain aspects, the RT-Cas1 fusion protein comprises an amino acid sequence at least 85%, 90%, 91%, 92%, 93%, 94%, 95%, 96%, 97%, 98%, 99% or 100% identical to SEQ ID NO: 3. SEQ ID NO: 3, the CRISPR-associated protein Cas1 from Marinomonas mediterranea (NCBI Reference Sequence: WP_013659858.1; 957 amino acids), is provided below (and which includes the Cas6, RT and Cas1 domains):

| | |
|---|---|
| 1 | mlnsplidav lplrsvvitl rwlspsktgf lhhaglhawv rflagspeqf sdfivvepie |
| 61 | nghisyqagd gyrfritvln ggeslldtlf sslkrlpesa anhpdiagaf sdnlvlekie |
| 121 | dtfehhqvtg iedlsvfdin almletavws rqrrfkvafn tparlvkpkp edgtelkgqn |
| 181 | rycrdksdln wqlfthrltd tfinlfqsrt gerlqrqnwp eaqlhaglav wlnnsytnkk |
| 241 | ekkvkdasgm laqmqieidd dfpadllall vlggyigmgq nrafgmgqyq lqdaygycsy |
| 301 | prpqaaksll ekslsdaslh qacqtmyprq anfdssdtde ehhdaidell tklyvsreri |
| 361 | fkreftpsql hsveiekpeg gtrllsvpnw hdrtlqkavt eclgntlehi wmkhsygyrk |
| 421 | ghsrlgardq ingyiqqgye wvlesdiesf fdsvnwlnle qr1klllpne plvpllmqwv |
| 481 | saakqtedeq tlarhnglpq gapispilan lllddldqdm iakghqivry addfvllfks |
| 541 | kaaaesaldd iitalkehhl ainlektriv easqgfrylg ylfvdgyaie tkreyrkeha |
| 601 | qldkqlnass lenepslqqe pavgnegstl igereklgtl liiagdiaml ssekqrlive |
| 661 | qydelhtypw atlssvllvg phhittpalk samfhnvpvh fasqygryqg vsagaapsvf |
| 721 | gadfwllqaq ylqqetnaln isqvliqari egiravisrr ekdapelnki grldekrlra |
| 781 | etldqlrgye gqaskqlwaf fqrileedwg ftgrnrrppk dpinallslg ytylyslvds |
| 841 | vnrtvglypw qgalhqrhgy hhtlasdlme pwrylvehvv ltlinrhqih kddfvikeng |
| 901 | cemssgarkt llkellvqlt kvpkggnsll temsnqsyrl alsckmqqrf iawspkr |

In further aspects, the RT-Cas1 fusion protein comprises an amino acid sequence at least 85%, 90%, 91%, 92%, 93%, 94%, 95%, 96%, 97%, 98%, 99% or 100% identical to SEQ ID NO: 5 (which includes the RT and Cas1 domains):

tklyvsreri
fkreftpsql hsveiekpeg gtrllsvpnw hdrtlqkavt eclgntlehi wmkhsygyrk
ghsrlgardq ingyiqqgye wvlesdiesf fdsvnwlnle qrlklllpne plvpllmqwv
saakqtedeq tlarhnglpq gapispilan lllddldqdm iakghqivry addfvllfks
kaaaesaldd iitalkehhl ainlektriv easqgfrylg ylfvdgyaie tkreyrkeha
qldkqlnass lenepslqqe pavgnegstl igereklgtl liiagdiaml ssekqrlive
qydelhtypw atlssvllvg phhittpalk samfhnvpvh fasqygryqg vsagaapsvf
gadfwllqaq ylqqetnaln isqvliqari egiravisrr ekdapelnki grldekrlra
etldqlrgye gqaskqlwaf fqrileedwg ftgrnrrppk dpinallslg ytylyslvds
vnrtvglypw qgalhqrhgy hhtlasdlme pwrylvehvv ltlinrhqih kddfvikeng
cemssgarkt llkellvqlt kvpkggnsll temsnqsyr1 alsckmqqrf iawspkr

In still further aspects, a RT polypeptide for use according to the embodiments comprises an amino acid sequence at least 85%, 90%, 91%, 92%, 93%, 94%, 95%, 96%, 97%, 98%, 99% or 100% identical to SEQ ID NO: 6:

tklyvsreri
fkreftpsql hsveiekpeg gtrllsvpnw hdrtlqkavt eclgntlehi wmkhsygyrk
ghsrlgardq ingyiqqgye wvlesdiesf fdsvnwlnle qrlklllpne plvpllmqwv
saakqtedeq tlarhnglpq gapispilan lllddldqdm iakghqivry addfvllfks
kaaaesaldd iitalkehhl ainlektriv easqgfrylg ylfvdgyai

In still further aspects, a Cas1 polypeptide for use according to the embodiments comprises an amino acid sequence at least 85%, 90%, 91%, 92%, 93%, 94%, 95%, 96%, 97%, 98%, 99% or 100% identical to SEQ ID NO: 7:

tl liiagdiaml ssekqrlive
qydelhtypw atlssvllvg phhittpalk samfhnvpvh fasqygryqg vsagaapsvf
gadfwllqaq ylqqetnaln isqvliqari egiravisrr ekdapelnki grldekrlra
etldqlrgye gqaskqlwaf fqrileedwg ftgrnrrppk dpinallslg ytylyslvds
vnrtvglypw qgalhqrhgy hhtlasdlme pwrylvehvv ltlinrhqih kddfvikeng
cemssgarkt llkellvqlt kvpkggnsll temsnqsyrl alsckmqqrf iawspkr

In further aspects, the RT, Cas1 or RT-Cas1 fusion protein is recombinant. In some aspects, the reverse transcriptase is a thermostable reverse transcriptase. In certain aspects, the thermostable reverse transcriptase comprises a bacterial reverse transcriptase. In some aspects, the reverse transcriptase comprises a group II intron or group II intron-like reverse transcriptase. In further aspects, a Cas1 and/or RT are fused to a purification/stabilization tag. In some aspects, the RT and Cas1 are fused and comprise a linker peptide between the RT and Cas1 domains. In certain aspects, the linker peptide is a non-cleavable linker peptide. In some embodiments, the linker peptide consists of 1 to 20 amino acids, while in other embodiments the linker peptide consists of 1 to 5 or 3 to 5 amino acids. For example, a rigid non-cleavable linker peptide can include 5 alanine amino acids.

In some aspects, the method further comprises providing Cas2. In some aspects, the Cas2 is bacterial Cas2. In certain aspects, the Cas2 is recombinant. In particular aspects, the Cas2 is provided as a RT-Cas1-Cas2 recombinant vector. In some aspects, the Cas2 comprises an amino acid sequence at least 80% identical to SEQ ID NO: 4. In certain aspects, the Cas2 protein comprises an amino acid sequence at least 85%, 90%, 91%, 92%, 93%, 94%, 95%, 96%, 97%, 98%, 99% or 100% identical to SEQ ID NO: 4. SEQ ID NO: 4, the CRISPR-associated protein Cas2 from Marinomonas mediterranea (NCBI Reference Sequence: WP_013659857.1; 92 amino acids), is provided below:

| | |
|---|---|
| 1 | mriylacfdi eddkkrrkls nllleygdry gysvfeislk denelhklrk kcskyteead |
| 61 | slrfywlnke srkhsgdvwg npiavfpaav |

In certain aspects, the dsDNA substrate comprises a CRISPR array or fragment thereof. For example, the CRISPR array is CRISP03. In some aspects, the Cas1 recognition sequence comprises at least one CRISPR repeat sequence and/or leader sequence. In certain aspects, the Cas1 recognition sequence comprises 2, 3, 4, or 5 CRISPR repeat sequences. For example, the CRISPR repeat sequence can comprise SEQ ID NO: 1 GTTTCAGACCCGCTGGCCGCTTAGGCCGTTGAGAC.

In some aspects, the CRISPR array comprises a leader sequence. In some aspects, leader sequence comprises SEQ ID NO: 2-TTGGAAAAAATAAGGGTACT, the sequence shown in FIG. 7 or SEQ ID NO: 7:

TAAACCCTTTATCAGTGAATAAACGATTTTTGCTCTTTAAAAACATAACC
TTAAAACAGTCCTCAATTGATTGAAGGGGTTTAGGGCGCGTTTTACATAA
AAATCAAAAACTTAGCTTGAAATAATGGCGAAAATTCACTAATTTTAAGC
ATACCTCTTGTGGATAACTTGAGGGCGGGGGAAACGCTAGGTTAACCTGC
TGAAATGATTGGAAAAAATAAGGGTACT.

For example, in some aspects, the CRISPR array on the dsDNA substrate comprises at least 5, 10, 15, 20, 25, 30, 35, 40, 45, 50, 55, 60, 65, 70, 75, 80, 85, 90, 95, 100, 125, 150, 175 or 200 nucleotides of SEQ ID NO: 7. In some aspects, the sequence comprises a fragment of SEQ ID NO: 7 that includes the sequence of SEQ ID NO: 2. In some aspects, the CRISPR array comprises a leader sequence, at least one repeat and a native spacer. In some aspects, the CRISPR array comprises a leader sequence, at least two repeat sequences and at least one native spacer. In some aspects, the at least one native spacer is a fragment of the native spacer. Accordingly, in some aspects, the RT-Cas1 and Cas2 protein complex cleaves the dsDNA substrate at the junction between the leader and the first repeat on the top strand and between the first repeat and spacer on the bottom strand. In some aspects, Cas1 produces a staggered cut in the DNA substrate. In some aspects, the dsDNA substrate further comprises a reporter.

In some aspects, the method further comprises the addition of CRISPR-associated factors. For example, the CRISPR-associated factors could be Cmr1, Cmr2, Cmr3, Cmr4, Cmr5, Cmr6, Marme_0670, and/or Marme_0671. In certain aspects, the CRISPR-associated factors may be provided in an expression construct.

In certain aspects, the method further comprises the addition of deoxynucleotide triphosphates (dNTPs). For example, the dNTPs are deoxyguanosine triphosphates (dGTPs) or deoxyadenosine triphosphates (dATPs).

In some aspects, the reverse transcriptase synthesizes DNA complementary to the ligated ssRNA of the RNA-DNA hybrid. In some aspects, the method further comprises deoxynucleotide triphosphates (dNTPs) to enable reverse transcription of the ligated RNA polynucleotide.

In some aspects, the method is performed in a host cell, such as a eukaryotic cell or a bacterial cell. In particular aspects, the host cell is comprised in an organism. In some aspects, providing the Cas1 polypeptide comprises providing an expression vector that encodes the Cas1 polypeptide. Thus, in certain aspects, the dsDNA substrate is provided to the host cell comprising at least a first polynucleotide or a population of polynucleotides. In some aspects, the host cell does not comprise one or more CRISPR system components, thus, the method further comprises providing one or more components of a CRISPR system to the host cell prior to or concomitant with providing the Cas1, such as the RT-Cas1, particularly an expression vector provided herein encoding the RT-Cas1 fusion protein.

In particular aspects, the host cell comprises one or more polynucleotides which are exogenous to the host cell, such as exogenous ssRNA. In some aspects, the exogenous RNA is derived from an infectious pathogen, such as viral, bacterial, or fungal RNA.

In some aspects, the method further comprises performing PCR amplification or sequencing of the dsDNA substrate comprising the integrated polynucleotide. In certain aspects, the method further comprises analyzing the results of the PCR amplification or sequencing to create a record of interactions of the host cell with exogenous RNA over time or to monitor the host cell's transcription profile over a period of time.

A further embodiment of the present disclosure provides a method for ligating RNA to DNA comprising: (a) obtaining ssRNA, dNTPs, and a target DNA comprising a Cas1 recognition sequence; and (b) providing a RT-Cas1 fusion protein, thereby producing a RNA-DNA hybrid. In some aspects, the assay is performed in vivo, such as in a host cell, particularly a bacterial or eukaryotic cell, such as a human cell. In some aspects, the host cell is comprised in an organism. In other aspects, the assay is performed in vitro.

In some aspects, the RT-Cas1 fusion protein is a bacterial RT-Cas1 fusion protein. In certain aspects, the bacterium is Arthrospira platensis or Marinomonas mediterranea.

In some aspects, the ssRNA has a length of about 10-100 nucleotides or any length derivable thereof, such as 20, 30, 40, 50, 60, 70, 80, or 90 nucleotides. In certain aspects, the ssRNA has a length of about 20-50 nucleotides. In particular aspects, the ssRNA is about 34, 35, or 36 nucleotides. In some aspects, the method comprises the addition of a population of ssRNAs. In some aspects, the population of ssRNAs comprises ssRNAs of a varying lengths. In certain aspects, the population of ssRNAs comprises 2, 3, 4, 5, 6, 10, 10², 10³, 10⁴, 10⁵, 10⁶, or 10⁷ ssRNAs. In some aspects, long RNA fragments are chemically sheared such as by heat and divalent metal cations to produce the population of ssRNAs. In other aspects, long RNA fragments are enzymatically or mechanically sheared to produce the population of ssRNAs.

In certain aspects, the dsDNA substrate comprises a CRISPR array or fragment thereof. For example, the CRISPR array is CRISP03. In some aspects, the Cas1 recognition sequence comprises at least one CRISPR repeat sequence. In certain aspects, the Cas1 recognition sequence comprises 2, 3, 4, or 5 CRISPR repeat sequences. For example, the CRISPR repeat sequence can comprise SEQ ID NO:1 GTTTCAGACCCGCTGGCCGCTTAGGCCGTTGAGAC.

In some aspects, the CRISPR array comprises a leader sequence. In some aspects, leader sequence comprises SEQ ID NO:2 CTGAAATGATTGGAAAAAATAAGGGTACT. In some aspects, the CRISPR array comprises a leader sequence, at least one repeat and a native spacer. In some aspects, the CRISPR array comprises a leader sequence, at least two repeat sequences and at least one native spacer. Accordingly, in some aspects, the RT-Cas1 and Cas2 protein complex cleaves the dsDNA substrate at the junction between the leader and the first repeat on the top strand and between the first repeat and spacer on the bottom strand. In some aspects, Cas1 produces a staggered cut in the DNA substrate. In some aspects, the dsDNA substrate further comprises a reporter.

In some aspects, the method further comprises the addition of CRISPR-associated factors. For example, the CRISPR-associated factors could be Cmr1, Cmr2, Cmr3, Cmr4, Cmr5, Cmr6, Marme_0670, and/or Marme_0671. In certain aspects, the CRISPR-associated factors are provided in an expression vector.

In certain aspects, the method further comprises detection of the integrated polynucleotide. In some aspects, the detection comprises performing PCR such as by primers to the CRISPR leader sequence and the first native spacer. In other aspects, the detection is performed by sequencing.

In some aspects, a population of polynucleotides is added to the dsDNA substrate and combined with Cas1. For example, a population of short RNA fragments is combined with the dsDNA substrate to create a DNA-RNA hybrid. In some aspects, the DNA-RNA hybrid is filled-in by using the reverse transcriptase activity of the RT-Cas1 fusion protein in the complex.

In another embodiment, the methods of the present disclosure can be used to produce an RNA expression library. In some aspects, the RT-Cas1 system is used to create a permanent record in the genome of a host of interactions with foreign RNA over a period of time. In other aspects, the RT-Cas1 system is used to monitor the transcription profile of an organism over time. In some aspects, the dsDNA substrate target of RT-Cas1 is provided to the host.

In certain aspects, the reverse transcriptase is HIV-1 RT, a group II intron RT or a a group II intron-like RT. Examples of thermostable bacterial reverse transcriptases include Thermosynechococcus elongatus reverse transcriptase and Geobacillus stearothermophilus reverse transcriptase. In another embodiment, the thermostable reverse transcriptase exhibits high fidelity cDNA synthesis. In some aspects, the thermostable reverse transcriptase is a Thermosynechococcus elongatus (Te) RT, Geobacillus stearothermophilus (Gs) RT, modified forms of these RTs, engineered variants of Avian myoblastosis virus (AMV) RT, Moloney murine leukemia virus (M-MLV) RT, or Human immunodeficiency virus (HIV) RT.

Another embodiment provides an isolated population of polynucleotides comprising a population of DNA-RNA chimeric molecules, each molecule comprising: (i) a first dsDNA region; (ii) a DNA/RNA region comprising one RNA strand and a complimentary DNA strand; and (iii) a second dsDNA region. In some aspects, the DNA/RNA region is 10-100 nucleotides in length. In certain aspects, the DNA/RNA region is 20-60 nucleotides in length. In some aspects, the population is substantially free of supercoiled DNA. In certain aspects, the first and second dsDNA region together comprise a Cas1 recognition sequence.

In a further embodiment, there is provided a method for reverse transcription of a target RNA to provide a complementary DNA comprising: (a) obtaining a target RNA; and (b) providing a RT-Cas1 protein, thereby providing the complementary DNA. In some aspects, the method is performed in the presence of added dNTPs. In some aspects, RT-Cas1 protein is from Arthrospira platensis or Marinomonas mediterranea. In certain aspects, the target RNA is comprised in a RNA-DNA chimeric molecule.

In a further embodiment, the methods of present disclosure provide methods of monitoring the transcription profile of a host or exposure to environmental pathogens. In some aspects, the RT-Cas1 protein complex is expressed in an organism to record events of pathogens infecting the organism in a permanent manner that allows analysis of rare events. In other aspects, the RT-Cas1 protein complex is used to generate a cumulative transcriptional profile of the organism over a determined period of time.

In some aspects, the host cell already comprises a CRISPR system and the CRISPR array polynucleotide which is introduced into the cell comprises the identical CRISPR array repeat sequence which is endogenous to that bacteria. In other aspects, the host cell does not comprise a CRISPR system and it will be appreciated that any CRISPR array may be introduced into the cell. According to this embodiment, the other components which make up the CRISPR system are also introduced into the cell. Such components typically match the CRISPR array (i.e. originate from the same CRISPR system). The other components may be introduced into the cell (together with a non-modified, native spacer, or on their own) prior to administration of the CRISPR array with the modified spacer. Alternatively, the other components may be introduced into the cell concomitant with (on the same or on a separate vector) the CRISPR array with the modified spacer.

In some aspects, the polynucleotides of the present disclosure are inserted into nucleic acid constructs so that they are capable of being expressed and propagated in host cells. In certain aspects, the nucleic acid constructs comprise a prokaryotic origin of replication and other elements which drive the expression of the CRISPR array and associated cas genes. In particular aspects, the promoter utilized by the nucleic acid construct is active in the specific cell population transformed. Constitutive promoters suitable for use with the present invention are promoter sequences which are active under most environmental conditions and most types of cells such as the cytomegalovirus (CMV) and Rous sarcoma virus (RSV). In some aspects, the promoter is an inducible promoter, i.e., a promoter that induces the CRISPR expression only in a certain condition (e.g. heat-induced promoter) or in the presence of a certain substance (e.g., promoters induced by Arabinose, Lactose, IPTG etc).

In yet another embodiment, there is provided an expression construct comprising a sequence encoding a RT and a Cas1 polypeptide or encoding a RT-Cas1 fusion protein. In some aspects, the RT-Cas1 fusion protein is a bacterial RT-Cas1 fusion protein. For example, the bacterial RT-Cas1 fusion protein is from Arthrospira platensis or Marinomonas mediterranea. In particular aspects, the RT-Cas1 fusion protein comprises an amino acid sequence at least 80% identical to SEQ ID NO: 3 or 5. In further aspects, the RT-Cas1 fusion protein comprises an amino acid sequence at least 85%, 90%, 91%, 92%, 93%, 94%, 95%, 96%, 97%, 98%, 99% or 100% identical to SEQ ID NO:3 or 5. In further aspects, the expression construct further comprises a sequence encoding a CRISPR adaptation gene. As used herein a “CRISPR adaptation gene” refers to a sequence encoding a factor that aides in CRISPR leader and/or CRISPR repeat acquisition. In particular aspects, the CRISPR adaption gene is Marme_0670.

In additional aspects, an expression construct (or method) of the embodiments further comprises a gene encoding for a Cas2 protein. In some aspects, the gene encoding for Cas2 protein encodes a Cas2 protein comprising an amino acid sequence at least 80% identical to SEQ ID NO: 4. In certain aspects, the gene encoding for Cas2 protein encodes for a Cas2 protein comprising an amino acid sequence at least 85%, 90%, 91%, 92%, 93%, 94%, 95%, 96%, 97%, 98%, 99% or 100% identical to SEQ ID NO: 4. In some aspects, the construct further comprises a reporter gene, such as GFP.

In some aspects, an expression construct (or method) of the embodiments further comprises providing a gene encoding a CRISPR array, such as a CRISP03 array. In specific aspects, a method comprises expressing a gene encoding the RT-Cas1 fusion protein and expressing CRISPR adaptation gene. In some aspects, the RT-Cas1 fusion protein and/or the CRISPR adaptation gene are under the control of a heterologous promoter. For example, the RT-Cas1 fusion protein and/or the CRISPR adaptation gene can be under the control of a first promoter (e.g., the parA promoter) and a CRISP03 array can be under the control of a second promoter (e.g., the pTrc promoter).

In other aspects, the RT-Cas1 fusion is recombinant. In some aspects, the RT is a thermostable reverse transcriptase. In certain aspects, the RT is a group II intron or group II intron-like reverse transcriptase. In some aspects, the Cas1 and RT are fused with a linker peptide. For example, the linker peptide can be a cleavable or a non-cleavable linker.

A further embodiment provides a RT-Cas1 fusion protein encoded by an expression construct provided herein. Further provided is a host cell comprising an expression construct provided herein as well as the RT-Cas1 fusion protein encoded by the expression construct.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1C: Phylogenetic distribution and domain structure of RT-Cas1 fusion proteins. (A) Taxonomic summary of unique RT-Cas1 protein records obtained from the NCBI CDART engine (current as of May, 2015). Shown are numbers of Cas1 protein records and bacterial species with (left) a fused RT domain, (center) RT and an additional N-terminal extension containing a Cas1-like motif, and (right) Cas1 with no additional annotated domain. Only phyla containing RT-Cas1 fusions are listed. (B) 16S rRNA-based tree showing major bacterial phyla, with phyla that contain RT-Cas1 including Cyanobacteria, Actinobacteria, Planctomycetes, Chlorobi, Bacteroidetes, and Proteobacteria (adapted from Ludwig and Klenk, 2001). (C) Schematic showing the domain organization of HIV RT (SwissProt P03366), a group II intron RT (TeI4c from T elongatus BP-1; Genbank WP_011056164), A. platensis RT-Cas1 (WP_006620498), M mediterranea RT-Cas1 (WP_013659858), and E. coli Cas1 (NP 417235). Conserved RT motifs as defined in (Xiong and Eickbush, 1990) are labeled 1 to 7. Motifs 0 and 2 a are conserved in mobile group II intron and non-LTR-retrotransposon RTs (Blocker et al., 2005). The YXDD sequence found in motif 5 contains two Asp residues at the RTactive site. Three a-helices found in the thumb/X domain of HIV and group II intron RTs are indicated. Numbers below the bars indicate amino acid positions. D, DNA binding domain; En, endonuclease domain.

FIGS. 2A-2F: Spacer acquisition in E. coli by ectopic expression of MMB-1 type III-B CRISPR components. (A) The MMB-1 type III-B CRISPR operon consists of an 8-spacer CRISPR array (CRISP03), followed by a canonical six-gene cassette putatively encoding the type III-B Cmr effector complex, two genes of unknown function (Marme_0671 and Marme_0670), the genes encoding RT-Cas1 and Cas2, and lastly a larger 58-spacer CRISPR array (CRISP0 2). The locus is flanked by two ˜200-bp direct repeats (small arrows). The black arrows indicate promoters. (B) Arrangement of MMB-1 type III-B CRISPR components under inducible promoters on pBAD (Para, Ptrc, and Plac) vectors for ectopic expression in E. coli . (C) Spacer detection frequency after overnight induction of E. coli carrying pBAD expression vectors with arabinose and IPTG. Wild-type RT-Cas1, RT active site mutant (YAAA), and Cas1 domain mutants E790A and E870A were tested with or without the Plac-driven gene cassette encoding the Cmr effector complex. Cas2 Δ32-92 and RT domain Δ299-588 mutants (shown in the two rightmost columns) were tested without the Cmr cassette. Bars indicate values for two biological replicates (means±SEM; n.d., not determined). (D) Histogram showing normalized counts of E. coli genomic protospacers from the wild-type RT-Cas1 and RTD spacer acquisition experiments, distributed by mappable length. Pooled data from several experiments are presented. (E) Nucleotide probabilities at each position along the wild-type RT-Cas1—acquired protospacers in (D), including 15 bp of flanking sequence on each side. Because of varying protospacer lengths, two panels are shown with the spacer 5′ and 3′ ends anchored at positions 15 and 35, respectively. (F) Cumulative normalized distribution of spacers in (D) among E. coli protein-coding ORFs sorted by expression level [normalized RNAseq read counts from (Haas et al., 2012); FPKM, fragments per kilobase permillion reads], with the most highly expressed genes listed first. Included are 2470 wild-type RT-Cas1- and 5569 RTΔ-acquired spacers mapping to E. coli genes (K12 genome). Dashed black lines show the range of values from a Monte Carlo simulation with random assortment (no transcription-related bias).

FIGS. 3A-3E: RT-Cas1-mediated spacer acquisition in MMB-1. (A) Arrangement of genes encoding Marme_0670, RT-Cas1, and Cas2 on pKT230 broad-host-range vectors under the control of the putative 16S rRNA promoter (P16S; 100-bp sequence upstream of the MMB-1 16S rRNA gene) for overexpression in MMB-1. New spacers were amplified from the genomic CRISP03 array. (B) Spacer detection frequency after overnight growth ofMMB-1 transconjugants carrying pKT230 overexpression vectors. Two clones each from two independent conjugations carrying either wild-type RT-Cas1, Cas1 domain mutants E790A or E870A, RT domain D299-588 mutants, or an empty pKT230 vector were tested. Bars depict spacer acquisition frequencies for two transconjugants (means±SEM). (C) Histogram showing normalized counts of MMB-1 genomic protospacers from the wild-type RT-Cas1 and RTD spacer acquisition experiments, distributed by mappable length. Pooled data from several experiments are presented. (D) Nucleotide probabilities at each position along the wild-type RT-Cas1-acquired protospacers in (C), including 15 bp of flanking sequence on each side. Because of varying protospacer lengths, two panels are shown with the spacer 5′ and 3′ ends anchored at positions 15 and 35, respectively. (E) Cumulative distribution of spacers in (C) among MMB-1 genes sorted by RNAseq FPKM, with the most highly expressed genes listed first. Included are 455 wild-type RT-Cas1- and 341 RTΔ-acquired spacers mapping to MMB-1 genes. Guides are drawn along the x axis at top-10% and top-50% genes by expression level. Monte Carlo bounds were calculated as in FIG. 2F. rRNA genes have been excluded from this analysis because spacers were rarely acquired from rRNA.

FIGS. 4A-4C: Spacer acquisition from RNA in the MMB-1 type III-B system. (A) Spacers acquired from a host genome could conceivably originate from either RNA or DNA. To test for an RNA origin, we used an engineered self splicing transcript, which produces an RNA sequence junction that is not encoded by DNA. Bases that were mutated to provide flanking exon sequences favorable for td intron splicing were separated by the 393-bp intron in the DNA template. After transcription and splicing, the two exons were brought together to form a novel junction containing the identifying mutations. Newly acquired spacers that contain this exon-junction indicate spacer acquisition from an RNA target. (B) Alignments of some of the genome-contiguous spacers (top) and several newly acquired exon-junction-spanning spacers (bottom) to the genomic and split-gene sequences, respectively (double colons indicate insertion of the td intron). Bases mutated to facilitate td intron splicing are underlined in the genomic sequences. Identifying mutations are depicted as light gray bases, and the splice sites are indicated by triangles. The highlighted ssrA exon-junction-spanning spacer (bottom) is antisense to the spliced tmRNA and differs from a putative DNA template by the five expected mutations. (C) All unique spacers spanning the td intron splice site that did not carry the engineered mutations. The maximum number of mismatches (MM) when these spacers were mapped to the wild-type genomic locus is indicated. None of the identifying mutations were observed among these sporadic mismatches. The spacers in (B) were in addition to four spacers (one for the S15 and three for the ssrA construct) that align to the unspliced exon-intron junction and could have been derived from either DNA or (nascent) RNA.

FIGS. 5A-5G: Site-specific CRISPR DNA cleavage-ligation by the RT-Cas1-Cas2 complex. (A) Schematic of CRISPR DNA substrates and products of cleavage-ligation reactions. The substrate was a 268-bp DNA containing the leader (gray), the first two repeats (R1 and R2) and spacers (S1 and S2), and part of the third repeat (R3) of the MMB-1 CRISP03 array. Cleavages (arrowheads) occur at the boundaries of the first repeat with concomitant ligation of a DNA or RNA oligonucleotide (oligo) to the 3′ fragment, yielding products of the sizes shown. (B) Internally labeled CRISPR DNA and a 33-nt dsDNA were incubated with no protein (lane 1), RT-Cas1 (lane 2), Cas2 (lane 3), or a 1:2 mixture of RT-Cas1 and Cas2 (lane 4).The sizes of products determined from sequencing ladders in parallel lanes are indicated on the left. (C) Internally labeled CRISPR DNA was incubated with wild-type RT-Cas1 and Cas2 without (lane 1) or with a 21-nt RNA (lane 2), 35-nt RNA (lane 3), or 29-nt ssDNA (lane 4). (D) Internally labeled CRISPR DNA was incubated with wild-type RT-Cas1 plus Cas2 in the absence (lane 1) or presence of a 29-nt ssDNA with either a 3′ OH (lane 2) or a 3′ phosphate (lane 3). (E) Nuclease digestion of 5′-end-labeled RNA and DNA oligonucleotides ligated to CRISPR DNA. Ligation reactions were performed as in (C). After extraction with phenol-CIA and ethanol precipitation, the products were incubated with the indicated nucleases. An asterisk indicates that the sample was boiled to denature the DNA before adding the nuclease. (F) Ligation of 5′-end-labeled RNA and DNA oligonucleotides into CRISPR DNA by wild-type (WT) and mutant RT-Cas1 proteins. Lanes 1 and 6 show control reactions of internally labeled CRISPR with WT RT-Cas1 plus Cas2 and an unlabeled 35-nt ssRNA or 29-nt ssDNA oligonucleotide for comparison. Lanes 2 to 5 and 7 to 10 show reactions of unlabeled CRISPR DNA with 5′-end-labeled 35-nt ssRNA and 29-nt ssDNA, respectively, and WT, E870A, and RTA RT-Cas1 plus Cas2. All reactions were carried out in the presence of dNTPs. (G) Effect of dNTPs. In the gel on the left, internally labeled CRISPR DNA was incubated with WT RT-Cas1 plus Cas2 in the presence of a 29-nt ssDNA (lanes 1 and 2) or 35-nt ssRNA (lanes 3 and 4) in the absence (lanes 1 and 3) or presence of 1 mM dNTPs (1 mM each of dATP, dCTP, dGTP, and dTTP; lanes 2 and 4). In the gel on the right, internally labeled CRISPR DNA was incubated with WT RT-Cas1 plus Cas2 in the presence of a 35-nt ssRNA oligonucleotide in the absence (lane 10) or presence of different dNTPs (1 mM) as indicated (lanes 5 to 9). Dots (labeled 155+oligo and 148+oligo) indicate products resulting from cleavage and ligation of oligonucleotides at the junction of the leader and repeat 1 on the top strand and the junction of repeat 1 and spacer 1 on the bottom strand, respectively; dots (near the top and bottom of the gel) indicate products of the size expected for cleavage and ligation of the oligonucleotide at the junctions of the second CRISPR repeat.

FIGS. 6A-6B: cDNA synthesis using RNA ligated to CRISPR DNA. (A) Schematic showing the CRISPR DNA substrate and the expected products of cleavage and ligation (top) followed by TPRT of the ligated RNA oligonucleotide. cDNAs are shown as dashes with arrowheads indicating the direction of cDNA synthesis. (B) WT or mutant RT-Cas1 plus Cas2 proteins were incubated with 268-bp CRISPR DNA in the presence of 21-nt RNA oligonucleotide, labeled dCTP, and unlabeled dATP, dGTP, and dTTP. The WT RT-Cas1-Cas2 complex yields labeled bands of the sizes expected (148 and 155 nt plus oligonucleotide) for TPRT of the RNA oligonucleotide that is ligated site-specifically at opposite boundaries of the first CRISPR DNA repeat (R1, lane 8).The labeled products were not detected with the RT domain (RTΔ, lane 9) or Cas1 active site (E870A, lane 10) mutants, but a background of labeled products is apparent in the E870A lane due to the RT activity of the protein in the absence of cleavage and ligation (FIG. 16). Labeled products were not detected in the absence of the RNA oligonucleotide (lanes 3 to 6) or CRISPR DNA (lanes 11 and 12). Separate lanes from the same gel (lanes 1 and 2) show the positions of cleavage-ligation products for RT-Cas1 plus Cas2 with an internally labeled CRISPR DNA substrate. “None” indicates no protein added.

FIGS. 7A-7D: Acquisition of new spacers by wild-type RT-Cas1 in E. coli and M. mediterranea MMB-1. (A) Schematic showing the leader-proximal region of an expanded CRISP03 array amplified by PCR in our spacer-detection assay. The leader sequence was identified by directional RNA sequencing of MMB-1 to determine the polarity of the CRISPR arrays. RNAseq data also confirmed that mature crRNAs with 8-nt 5′-repeat-derived handles (17) were being generated. The native spacers in both CRISPR arrays in this system were 34-36 bp long and did not match any other sequence in GenBank. (B) Alignments of a subset of newly acquired spacers from ectopic E. coli assays to the dnaK and dnaJ genes. (C) Alignments of a subset of newly acquired spacers from MMB-1 overexpression assays to Marme_0568 and Marme_0569 (dnaK and dnaJ homologs respectively). Marme_0568 is ˜5 fold more highly expressed than Marme_0569 (RNAseq data from this study) and is sampled ˜20 times more frequently by the RT-Cas1 spacer acquisition machinery in MMB-1. (D) Total counts of newly acquired genomic and plasmid protospacers detected in all experiments with wild-type spacer acquisition components in E. coli and MMB-1.

FIGS. 8A-8C: RT-independent sense-strand bias in spacer acquisition by RT-Cas1 in MMB-1 but not E. coli . (A) Percentage of spacers from E. coli ectopic assay (data from FIG. 2D) acquired from coding and template strands of E. coli genes, and from intergenic regions (note that all regions not annotated as genes are considered intergenic for this analysis; a fraction of these are transcribed, e.g., intergenic sequences within operons). (B) Percentage of spacers isolated from the endogenous copy of MMB-1 CRISP03 (data from FIG. 3C) acquired from sense and antisense strands of MMB-1 genes, and from intergenic regions. The bias for the sense strand persists in the RtΔ-Cas1 acquired spacer pool. The larger dataset of spacers isolated from the plasmid-supplied copy of CRISP03 (data from FIG. 13C) exhibits a less pronounced bias for the coding strand; these data were collected using a modified spacer detection protocol for transconjugants with plasmid copies of CRISP03. (C) Cumulative distribution of spacers among MMB-1 genes sorted by RNAseq FPKM (RNAseq data from FIG. 3E), with most highly expressed genes listed first (note that these expression profiles were obtained from different MMB-1 transconjugants than FIG. 3E). Wild-type RT-Cas l-acquired spacers isolated from plasmid copies of CRISP03 (data from FIG. 13C) were split into two pools: 43,766 spacers mapping to the sense strand of MMB-1 genes, and 32,573 spacers mapping to the antisense strand. Monte Carlo bounds were calculated as in FIGS. 2F, 3E.

FIGS. 9A-9B: Protospacer sequence composition for RtΔ constructs. Nucleotide probabilities at each position along the protospacers acquired by the RTΔ version of RT-Cas1 in (A) E. coli , and (B) MMB-1, including 15 bp of flanking sequence on each side. Due to varying protospacer lengths, two panels are shown with spacer 5′ and 3′ ends anchored at positions 15 and 35, respectively.

FIG. 10: Proportion of genome and plasmid derived spacers in MMB-1. A total of 497 spacers mapping to the MMB-1 genome, and 24 to the pKT230 expression vector were recovered in experiments with MMB-1 strains where wild-type RT-Cas1 associated genes were overexpressed. DNA was sequences from one such transconjugant using Nextera technology (Illumina, Inc.) to measure the plasmid copy number and observed no enrichment for plasmid-derived spacers. Upon deletion of the RT domain of RT-Cas1, Nextera profiling of total DNA revealed that the plasmid copy number had remained unchanged, but the proportion of plasmid-derived spacers had increased 6-fold from 4.6% to 33% (369 spacers mapping to the MMB-1 genome and 181 to the pKT230 expression vector). In contrast, spacer acquisition by the native E. coli Cas1/Cas2 complex is 100-1000× biased towards plasmid DNA (Solano et al., 2000).

FIG. 11: Protospacer association with transcription level for RT active site mutant. Cumulative distribution of spacers among MMB-1 genes sorted by RNAseq FPKM (RNAseq data from FIG. 3E), with most highly expressed genes listed first (note that these expression profiles were obtained from different MMB-1 transconjugants and growth conditions than in FIG. 3E, in particular a lower incubation temperature: 23° C.). 3,631 wild-type RT-Cas1 , and 472 RT active site mutant (YAAA)-acquired spacers isolated from plasmid copies of CRISP03 mapping to MMB-1 genes are included. Monte Carlo bounds were calculated as in FIGS. 2F, 3E.

FIGS. 12A-12C: Verification of td intron splicing. (A) Electrophoresis of spliced and unspliced in vitro transcripts from td intron containing copies of the MMB-1 ribosomal protein S15 and ssrA tmRNA genes shows efficient splicing activity. All lanes have been cropped and placed together from the same gel. (B) Numbers of reads of spliced and unspliced transcripts in MMB-1 clones obtained from two independent conjugations (denoted 1 and 2) per construct, as determined by RT-PCR and high-throughput sequencing. (C) Numbers of reads from targeted DNA sequencing analyses of the same bacterial cultures used in (B) to empirically determine whether td exon-exon junctions are present in DNA form outside of the CRISPR locus.

FIGS. 13A-13E: RT-Cas1 mediated spacer acquisition into plasmid copies of CRISP03 in MMB-1. (A) Gene arrangement of MMB-1 expression constructs. To demonstrate spacer acquisition from RNA, a self-splicing td intron was inserted within plasmid copies of two genes that were frequently sampled by the spacer acquisition machinery—the gene encoding ribosomal protein S15, and the ssrA gene encoding tmRNA. The unstructured “mRNA like domain” of the tmRNA was chosen as it was highly over-represented in our initial spacer pools. Bases that were mutated to provide flanking exon sequences favorable for td intron splicing are depicted as colored bars within the exons of the intron-containing construct. (B) Spacer detection frequency from plasmid-encoded CRISP03 arrays using a modified spacer detection protocol (see Example 7), as compared with spacer acquisition into the endogenous CRISP03 array (data for the latter redrawn from FIG. 3B). Bars indicate values of two biological replicates for each td intron-containing construct. (C) Histogram showing normalized counts of MMB-1 protospacers isolated from plasmid copies of CRISP03, distributed by mappable length. Pooled data from several experiments are presented. (D) Nucleotide probabilities at each position along the wild-type RT-Cas1-acquired protospacers in (C) including 15 bp of flanking sequence on each side. Due to varying protospacer lengths, two panels are shown with spacer 5′ and 3′ ends anchored at positions 15 and 35, respectively. (E) Cumulative distribution of spacers in (C) among MMB-1 genes sorted by RNAseq FPKM (RNAseq data from FIG. 3E) with most highly expressed genes listed first (note that these expression profiles were obtained from different MMB-1 transconjugants than in FIG. 3E). 77,050 wild-type RT-Cas1-acquired spacers isolated from plasmid copies of CRISP03 mapping to MMB-1 genes are included and are distributed similarly to the 455 wild-type RT-Cas1 acquired spacers isolated from the endogenous CRISP03 array (data for the latter redrawn from FIG. 3E). Monte Carlo bounds were calculated as in FIGS. 2F, 3E.

FIGS. 14A-14B: MMB-1 RT-Cas1 is an active reverse transcriptase in vitro. (A) Wild-type (WT) and mutant RT-Cas1 proteins (1-2 μM final concentration) were assayed for RT activity by polymerization of radiolabeled dTTP in 30-min time courses using the artificial template-primer substrate poly(rA)/oligo(dT)24. The bar graphs show RT activity measured as moles of ³²P-dTTP polymerized per minute per mole protein, based on the initial rate of ³²P-dTTP incorporation and normalized to RT activity of WT RT-Cas1 assayed in parallel. Two independent protein preparations were assayed in duplicate. Wild-type RT-Cas1 protein has RT activity that is abolished by deletion of the RT domain (RtΔ) or mutations at the RT active site (YADD 4 YAAA at aa pos. 530-533). Note that the two Cas1 active site mutants, E790A and E870A, behave differently in RT assays: E870A has high RT activity comparable to that of the wild-type protein, but E790A has very little activity, suggesting interaction between the RT and Cas1 domains. (B) RT assays of WT RT-Cas1 with different template-primer substrates show that the putative RT activity requires both the poly(rA) template and oligo(dT) DNA primer, excluding terminal transferase activity, and that the wild-type protein also has some DNA-dependent DNA polymerase activity when assayed with poly(dA)/oligo(dT)₂₄. Error bars in (A) and (B) indicate standard deviations for at least 3 replicates in each case.

FIG. 15: CRISPR DNA cleavage and oligonucleotide ligation in vitro. Wild-type (WT) and mutant RT-Cas1 proteins with or without Cas2 were incubated with the internally labeled 268 bp CRISPR DNA and 33 -nt dsDNA (left), 29-nt ssDNA (middle), or 21-nt RNA (right) oligonucleotides in the absence (top panels) or presence (bottom panels) of dNTPs. RT-Cas1 has non-specific nuclease activity indicated by degradation products of the labeled CRISPR DNA in the absence of Cas2. The cleavage of CRISPR DNA and ligation of DNA oligonucleotides requires both Cas1 and Cas2. The RT mutations (RtΔ and YAAA) inhibit ligation of RNA but not DNA oligonucleotides, and dNTPs are required for ligation of RNA but not DNA oligonucleotides (also see FIG. 5). Dots and squares indicate the expected cleavage/ligation products as indicated in the schematic below. A larger band of unknown composition is seen above the 155-nt+oligo product in some lanes. The numbers to the left indicate the sizes of the CRISPR DNA cleavage and ligation products determined from a DNA sequencing ladder run in parallel lanes of the same gel. The schematic at the bottom shows the structure and size of the CRISPR DNA substrate and the cleavage-ligation products, with cleavage sites indicated by arrowheads. The products resulting from ligation of the DNA or RNA oligonucleotide to 5′ ends of the downstream fragments of both strands are indicated by light and dark circles, and the corresponding upstream fragments are indicated by light and dark squares.

FIG. 16: Schematic showing the products resulting from RT-Cas1 catalyzed cleavage-ligation reactions with the CRISPR DNA substrate. Cleavage and ligation at the 5′ ends of the first repeat junctions (black) produces 5′ fragments of 120 and 113 nt, and 3′ fragments of 148 and 155 nt plus the ligated oligonucleotides (dark and light dots). The same reaction at the 5′ ends of the second repeat produces 5′ fragments of 45 and 188 nt, and 3′ fragments of 80 and 223 nt plus the ligated oligonucleotide (dark and light dots). Labeled products of the expected size for cleavage and ligation at the second repeat junctions can be seen as weak bands in FIG. 5C, lane 4, FIG. 5E, lanes 6, 7, 9, and 10, and FIG. 5F, lanes 6, 8 and 9. Oligonucleotides of various sequences and sizes (ssDNA 19-59 nt; RNA 21-50 nt) can function as substrates for the cleavage/ligation reaction.

### DESCRIPTION OF ILLUSTRATIVE EMBODIMENTS

CRISPR systems mediate adaptive immunity in diverse prokaryotes. CRISPR-associated Cas1 and Cas2 proteins have been shown to enable adaptation to new threats in type I and II CRISPR systems by the acquisition of short segments of DNA (i.e., spacers) from invasive elements. In several type III CRISPR systems, Cas1 is naturally fused to a reverse transcriptase (RT). In the marine bacterium Marinomonas mediterranea (MMB-1), the inventors showed that a RT-Cas1 fusion protein enables the acquisition of RNA spacers in vivo in a RT-dependent manner. In vitro, the MMB-1 RT-Cas1 and Cas2 proteins catalyze the ligation of RNA segments into the CRISPR array, which is followed by reverse transcription. Accordingly, these observations outline a host-mediated mechanism for reverse information flow from RNA to DNA.

Thus, methods of the present disclosure overcome challenges associated with current technologies by providing an RT-Cas1 fusion protein to site-specifically ligate RNA and/or DNA to a target sequence in vivo or in vitro. In one method, the RT-Cas1 and Cas2 protein complex cleaves the CRISPR array site specifically at the junctions between the leader and first repeat on the top strand and between the first repeat and spacer on the bottom strand, producing a staggered cut. Concomitantly, short polynucleotides (e.g., 19-59 nt long, single-stranded or double-stranded RNA or DNA) are ligated covalently to the 3′ fragment of the CRISPR DNA. This produces a molecule that has, for example, a single stranded RNA attached to a short single stranded DNA followed by a segment of double-stranded DNA. This product allows for ‘filling-in’ the single stranded DNA-RNA hybrid by using the reverse transcriptase activity of the RT-Cas1 protein in the complex, and thus producing, for example, a labelled complementary molecule for further analysis.

In addition, the reverse transcriptase activity of the RT-Cas1 protein complex produces a DNA copy of any RNA ligated to the target DNA. This method improves on protein complexes that can only use double stranded DNA, and it also includes reverse transcriptase activity to produce cDNAs. Accordingly, the RT-Cas1 protein complex could be developed for use as a single-step RNAseq method for diagnostics, research and therapy. Additionally, it can be used for environmental monitoring of pathogens, and for general use as a reagent in molecular biology research.

### II. DEFINITIONS

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

By “expression construct” or “expression cassette” is meant a nucleic acid molecule that is capable of directing transcription. An expression construct includes, at a minimum, one or more transcriptional control elements (such as promoters, enhancers or a structure functionally equivalent thereof) that direct gene expression in one or more desired cell types, tissues or organs. Additional elements, such as a transcription termination signal, may also be included.

A “vector” or “construct” (sometimes referred to as a gene delivery system or gene transfer “vehicle”) refers to a macromolecule or complex of molecules comprising a polynucleotide to be delivered to a host cell, either in vitro or in vivo.

A “plasmid,” a common type of a vector, is an extra-chromosomal DNA molecule separate from the chromosomal DNA that is capable of replicating independently of the chromosomal DNA. In certain cases, it is circular and double-stranded.

An “origin of replication” (“ori”) or “replication origin” is a DNA sequence, e.g., in a lymphotrophic herpes virus, that when present in a plasmid in a cell is capable of maintaining linked sequences in the plasmid and/or a site at or near where DNA synthesis initiates. As an example, an ori for EBV includes FR sequences (20 imperfect copies of a 30 bp repeat), and preferably DS sequences; however, other sites in EBV bind EBNA-1, e.g., Rep* sequences can substitute for DS as an origin of replication (Kirshmaier and Sugden, 1998). Thus, a replication origin of EBV includes FR, DS or Rep* sequences or any functionally equivalent sequences through nucleic acid modifications or synthetic combination derived therefrom. For example, the present disclosure may also use genetically engineered replication origin of EBV, such as by insertion or mutation of individual elements, as specifically described in Lindner, et. al., 2008.

A “gene,” “polynucleotide,” “coding region,” “sequence,” “segment,” “fragment,” or “transgene” that “encodes” a particular protein, is a nucleic acid molecule that is transcribed and optionally also translated into a gene product, e.g., a polypeptide, in vitro or in vivo when placed under the control of appropriate regulatory sequences. The coding region may be present in either a cDNA, genomic DNA, or RNA form. When present in a DNA form, the nucleic acid molecule may be single-stranded (i.e., the sense strand) or double-stranded. The boundaries of a coding region are determined by a start codon at the 5′ (amino) terminus and a translation stop codon at the 3′ (carboxy) terminus. A gene can include, but is not limited to, cDNA from prokaryotic or eukaryotic mRNA, genomic DNA sequences from prokaryotic or eukaryotic DNA, and synthetic DNA sequences. A transcription termination sequence will usually be located 3′ to the gene sequence.

The term “promoter” is used herein in its ordinary sense to refer to a nucleotide region comprising a DNA regulatory sequence, wherein the regulatory sequence is derived from a gene that is capable of binding RNA polymerase and initiating transcription of a downstream (3′ direction) coding sequence. It may contain genetic elements at which regulatory proteins and molecules may bind, such as RNA polymerase and other transcription factors, to initiate the specific transcription of a nucleic acid sequence. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence.

The term “cell” is herein used in its broadest sense in the art and refers to a living body that is a structural unit of tissue of a multicellular organism, is surrounded by a membrane structure that isolates it from the outside, has the capability of self-replicating, and has genetic information and a mechanism for expressing it. Cells used herein may be naturally-occurring cells or artificially modified cells (e.g., fusion cells, genetically modified cells, etc.).

As used herein, “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

The terms “polypeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” includes natural and/or unnatural or synthetic amino acids, including glycine and both the D or L optical isomers, and amino acid analogs and peptidomimetics.

A “fusion protein,” as used herein, refers to a protein having at least two heterologous polypeptides covalently linked in which one polypeptide comes from one protein sequence or domain and the other polypeptide comes from a second protein sequence or domain.

The term “thermostable” refers to the ability of an enzyme or protein (e.g., reverse transcriptase) to be resistant to inactivation by heat. Typically such enzymes are obtained from a thermophilic organism (i.e., a thermophile) that has evolved to grow in a high temperature environment. Thermophiles, as used herein, are organisms with an optimum growth temperature of 45° C. or more, and a typical maximum growth temperature of 70° C. or more. In general, a thermostable enzyme is more resistant to heat inactivation than a typical enzyme, such as one from a mesophilic organism. Thus, the nucleic acid synthesis activity of a thermostable reverse transcriptase may be decreased by heat treatment to some extent, but not as much as would occur for a reverse transcriptase from a mesophilic organism. “Thermostable” also refers to an enzyme which is active at temperatures greater than 38° C., preferably between about 38-100° C., and more preferably between about 40-81° C. A particularly preferred temperature range is from about 45° C. to about 65° C.

### III. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Common Features of RT-Cas1 fusions

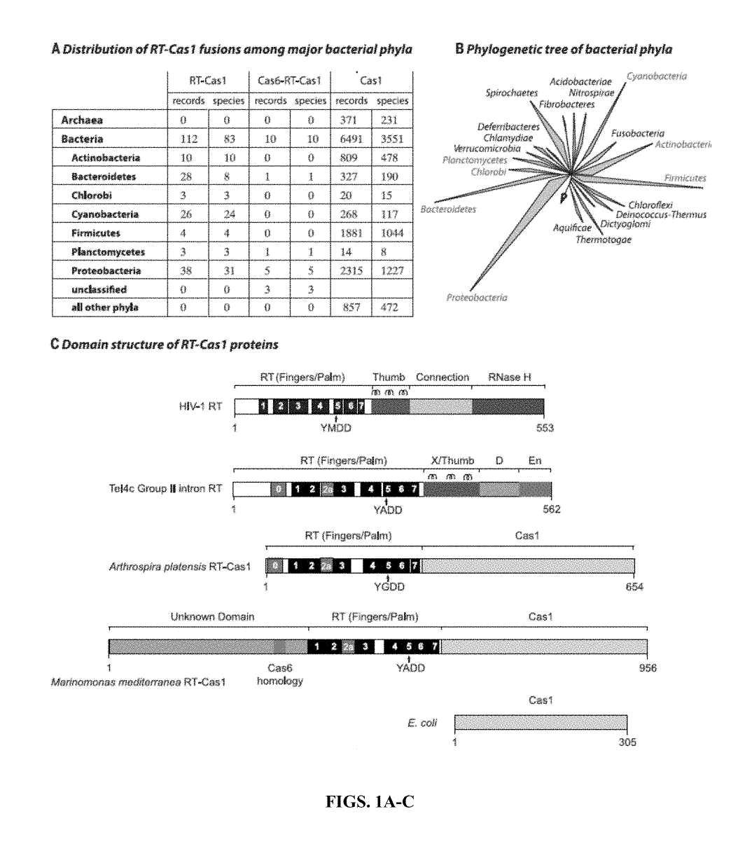

To examine the phylogenetic distribution of fused RT-Cas1-encoding genes, the National Center for Biotechnology Information (NCBI) Conserved Domain Architecture Retrieval Tool (CDART) was used to retrieve protein records containing both a Cas1 domain (Pfam database PF01867) and a RT domain of any origin (Pfam database PF00078). Of 93 RT-Cas1-bearing species, all were from bacteria and none were from archaea. RT-Cas1 fusions were most prevalent among cyanobacteria, with 21% of casl-bearing F1 cyanobacteria carrying such fusions (FIG. 1A and B). RT-Cas1 fusions with sufficient flanking sequence for type classification were exclusively associated with type III CRISPR systems; conversely, ˜8% of bacterial type III CRISPR systems carried RT-Cas1 fusions.

The Cas1-fused RT domains were most closely related to RTs encoded by mobile genetic elements (retrotransposons) known as mobile group II introns (Simon and Zimmerly, 2008; Toro and Nisa-Martínez, 2014). Two related structural families of RT-Cas1 proteins were identified. The more abundant family carries a canonical N-terminal RT domain with a conserved RT-0 motif characteristic of group II intron and non-long terminal repeat (LTR)-retrotransposon RTs (Malik et al., 1999; Blocker et al., 2005). This is likely also the case for MMB-1 RT-Cas1. The other group lacks the RT-0 motif, starting instead with an additional N-terminal domain containing a putative Cas6-like RNA recognition motif of the RAMP [repeat-associated mysterious protein (Makarova et al., 2006)] superfamily. Alignments of the retrovirus HIV-1 RT and a group II intron RT [Thermosynechococcus elongatus TeI4c RT (Mohr et al., 2013)] with representatives of the two RT-Cas1 fusion families (from Arthrospira platensis and Marinomonas mediterranea) revealed that both Cas1-fused RTs contain the seven conserved sequence motifs characteristic of the finger and palm regions of retroviral RTs. Each also shares the RT-2a motif, which is conserved in group II intron RTs and related proteins but not present in retroviral RTs, such as the HIV-1 RT (Malik et al., 1999; Blocker et al., 2005). The thumb/X domain, which is found in retroviral and group II intron RTs just downstream of the RT domain, appears to be missing in the Cas1-associated RTs (FIG. 1C).

The structural subcategories, limited phylogenetic distribution, and exclusive association with a subset of CRISPR types are consistent with a small number of common origins of RT-Cas1 fusions (Makarova et al., 2006; Simon and Zimmerly, 2008).

### Example 2

### Spacer Acquisition by the M. mediterranea Type III-B Machinery in an E. coli Host

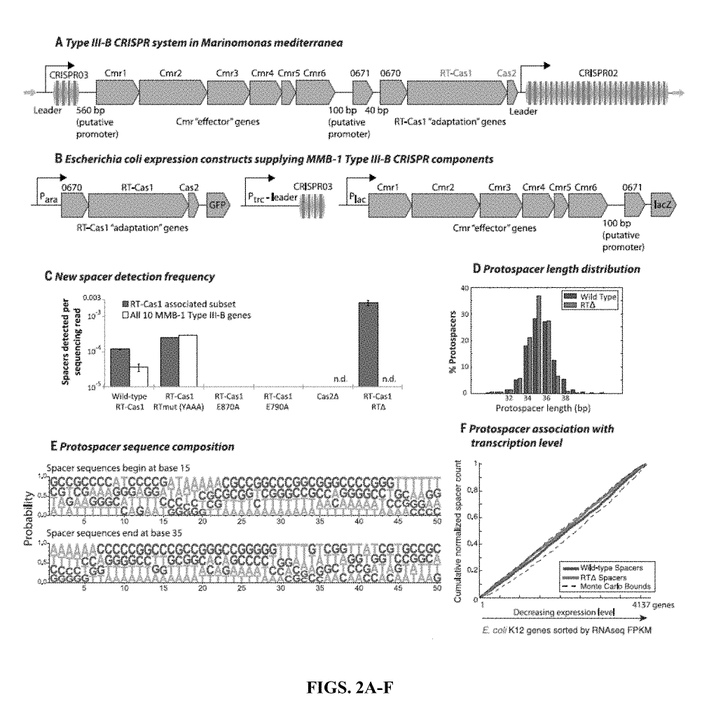

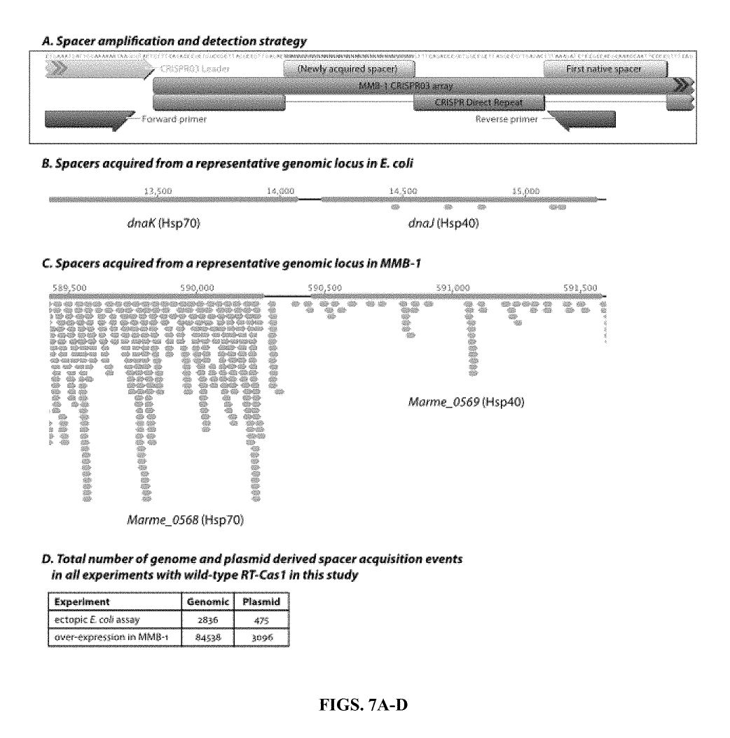

To test whether RT-Cas1 proteins could facilitate the acquisition of new spacers, and to determine whether such spacers might be acquired from RNA, the type III-B CRISPR locus in M. mediterranea (MMB-1) (Solano and Sanchez-Amat, 1999) was chosen, because this is an, easily cultured, nonpathogenic member of the well-studied γ-probacteriumium class that contains a RT-Cas1-encoding gene. Spacer acquisition was first assessed after transplantation of the locus into the canonical γ-probacteriumium experimental model, Escherichia coli. Expression vectors were constructed carrying the type III-B operon of MMB-1 in two configurations, either as a single cassette consisting of the CRISP03 array, the genes encoding RT-Cas1 and Cas2, and an adjacent gene (encoding Marme_0670) with limited homology to the NERD (nuclease-related domain) family (Grynberg et al., 2004), or together with a second cassette encoding the remaining CRISPR-associated factors, Cmr1 to Cmr6 and Marme_0671 (FIGS. 2A and 2B). The acquisition of new spacers into CRISP03 was evident from polymerase chain reaction (PCR) amplification of the region between the leader sequence and the first native spacer, followed by high-throughput sequencing. Newly acquired spacers were identified in transformants expressing either the full complement of Cas-encoding genes, or the subset containing only the potential “adaptation” genes (encoding RT-Cas1, Cas2, and Marme_0670). Bonafide spacer acquisition is evidenced by the precise junctions between the inserted spacer DNA and CRISPR repeats (FIG. 7A) and by the diversity of acquired spacers (FIG. 7B, 7D).

Specificity was further tested by evaluating the requirements for RT-Cas1 and Cas2 in spacer acquisition. Two point mutations, E870A and E790A, were constructed in the putative Cas1 active site of MMB-1 RT-Cas1 , based on a three-dimensional homology model computed using the Archaeoglobus fulgidus Cas1 crystal structure (Kim et al., 2013). Each point mutation abolished spacer acquisition, as did a 60-amino acid C-terminal deletion in Cas2 (FIG. 2C).

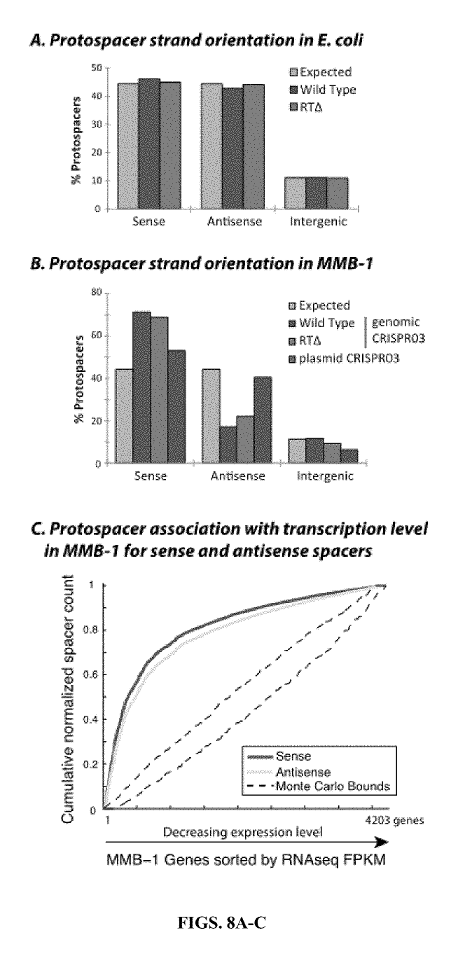

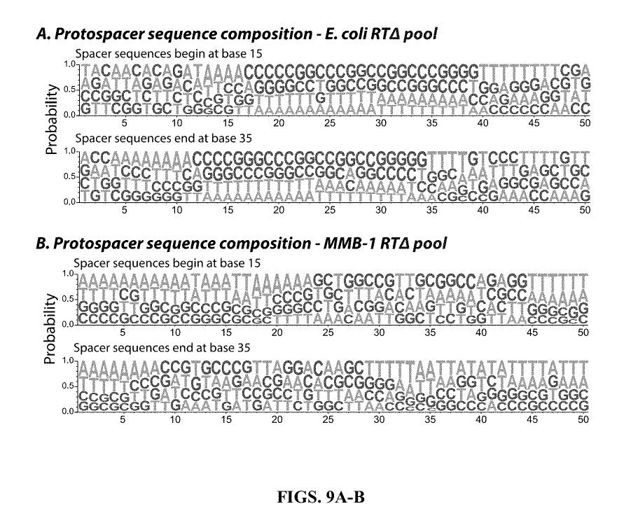

The majority (˜85%) of newly acquired spacers mapped to the E. coli genome, with the rest being derived from plasmid DNA (FIG. 7D). Over 70% of the spacers were 34 to 36 base pairs (bp) in length (FIG. 2D). Consistent with observations of interference mechanisms in other type III CRISPR systems (van der Oost et al., 2014), no evidence was found for a conserved protospacer-adjacent motif (PAM) or other sequence signature associated with protospacer choice (FIG. 2E). No bias was observed for the sense strand among spacers acquired from annotated E. coli genes (FIG. 8A) and no enrichment of spacers derived from highly transcribed genes (FIG. 2F). Spacer acquisition was unhindered when the RT domain of RT-Cas1 was mutated or deleted (FIG. 2C), consistent with a DNA-based mechanism operating under these conditions. Deletion of the entire 290-amino acid conserved region of the RT domain resulted in a ˜20-fold increase in spacer acquisition frequency, with no apparent differences in the characteristics of the pool of acquired spacers (FIGS. 2C, 2E, 2F, 8A and 9 A).

### Example 3

### Transcription-Associated Spacer Acquisition in MMB-1 is RT-Dependent

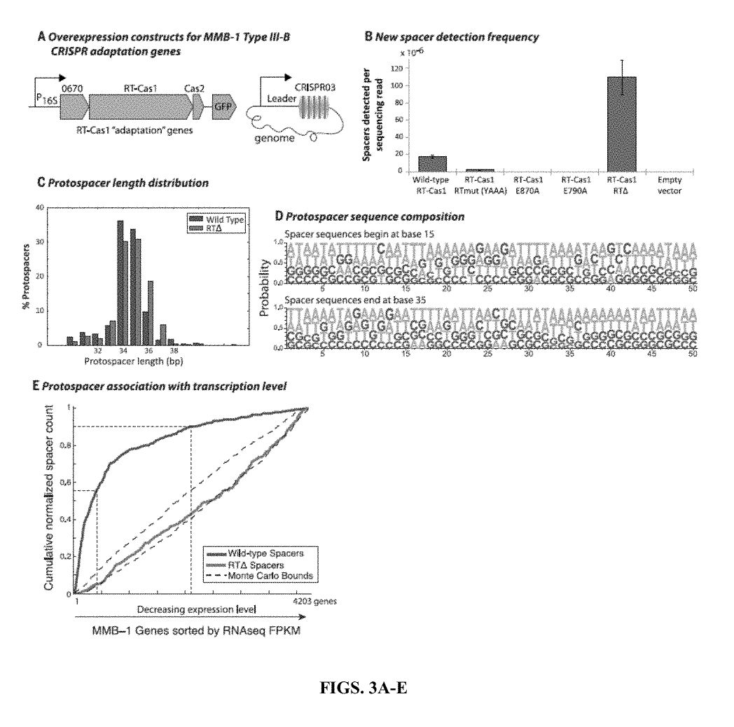

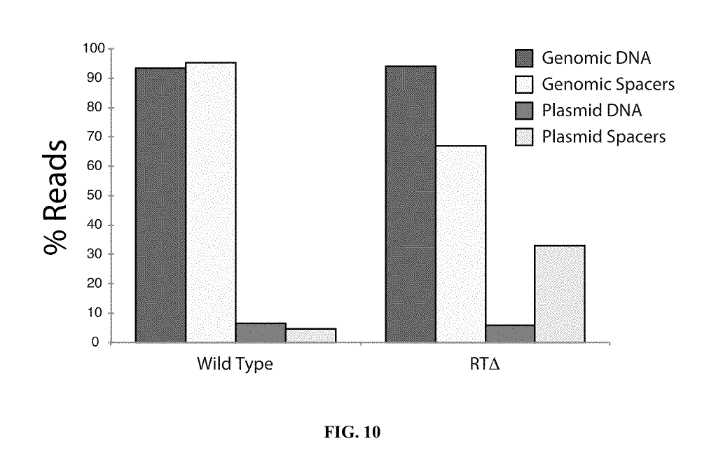

The inability to detect RNA spacer acquisition in the ectopic E. coli assay could reflect the absence of required factors or conditions that are present in the native host, MMB-1. To assay spacer acquisition in MMB-1, the RT-Cas1 and Cas2 open reading frames (ORFs) were overexpressed along with Marme 0670 from a broad-host-range plasmid (pKT230), using the 100-bp sequence upstream of the MMB-1 16S ribosomal RNA (rRNA) gene as a F3 promoter (FIG. 3A). Newly acquired spacers were recovered from the genomic copy of the CRISP03 array and it was found that the vast majority (˜95%) mapped to the MMB-1 genome, with an expected proportion mapping to the expression vector (FIGS. 7C, 7D and 10). Although the endogenous type III-B CRISPR operon was still present in these strains, it was found that plasmid-driven overexpression of adaptation genes was critical for detectable acquisition of new spacers. Parallel analysis of transconjugants in which plasmid-driven RT-Cas1 had the mutation E870A or E790A at the putative Cas1 active site, or of transconjugants carrying an empty vector, failed to identify any new spacers (FIG. 3B). As in E. coli , most (>75%) of the new protospacers were 34 to 36 bp in length (FIG. 3C), and no PAM-like sequences were observed at either the 5′ or 3′ ends of the acquired spacers (FIG. 3D).

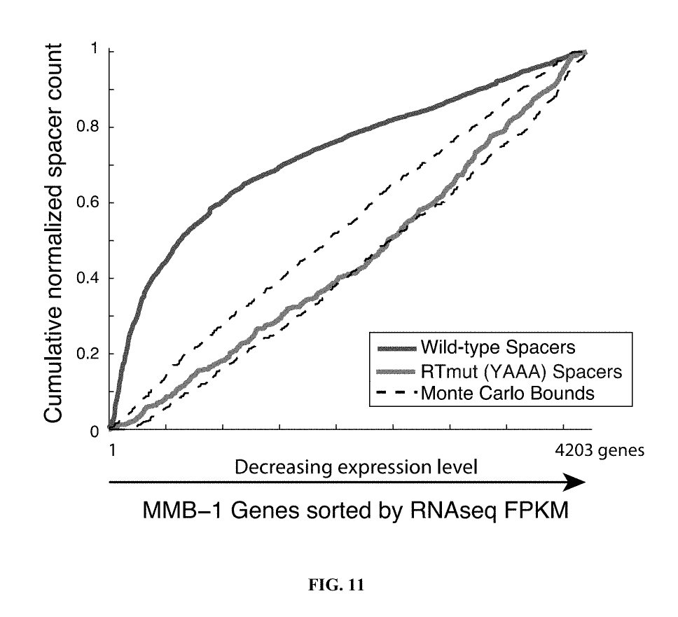

In contrast to the E. coli data set, the genomic regions most frequently sampled by the RT-Cas1 spacer acquisition machinery in MMB-1 appeared to be genes that are typically highly expressed in bacteria. This association was further investigated between expression and spacer capture by obtaining RNA sequencing (RNAseq) expression profiles of two independent MMB-1 transconjugants carrying the RT-Cas1 expression vector. The 10% most highly expressed genes accounted for over 50% of newly acquired spacers, with the top 50% of expressed genes accounting for 90% of newly acquired spacers (FIG. 3E). Next, it was tested whether this transcriptional association was dependent on the RT domain of RT-Cas1 . Deletion of the conserved RT domain of RT-Cas1 abolished the preference for highly transcribed genes (FIGS. 3E and 11), while maintaining a comparable length and sequence distribution for the acquired spacer repertoire (FIGS. 3B, 3C, 8B, 9B, and 10). Together, these data demonstrate a RT-dependent bias toward the acquisition of spacers from highly transcribed regions.

Spacers acquired from transcribed regions could conceivably be integrated into the CRISPR array in either a negative or a positive orientation. Among spacers that mapped to MMB-1 transcripts, there was observed at most a limited preference for the sense strand (FIGS. 8B and 8C). The lack of a strong bias implies a degree of directional flexibility in the integration mechanism, potentially yielding a system in which only a fraction of spacers is able to protect against a single-stranded DNA or RNA target.

### Example 4

### RT-Cas1-Mediated Spacer Acquisition from RNA

The observed association between the gene expression level and the frequency of spacer acquisition in MMB-1, combined with the requirement of the RT domain for this association, is consistent with an acquisition process involving reverse transcription of an RNA molecule. Nonetheless, an alternative hypothesis is that acquisition of DNA spacers could result from increased accessibility of DNA in regions of high transcriptional activity.

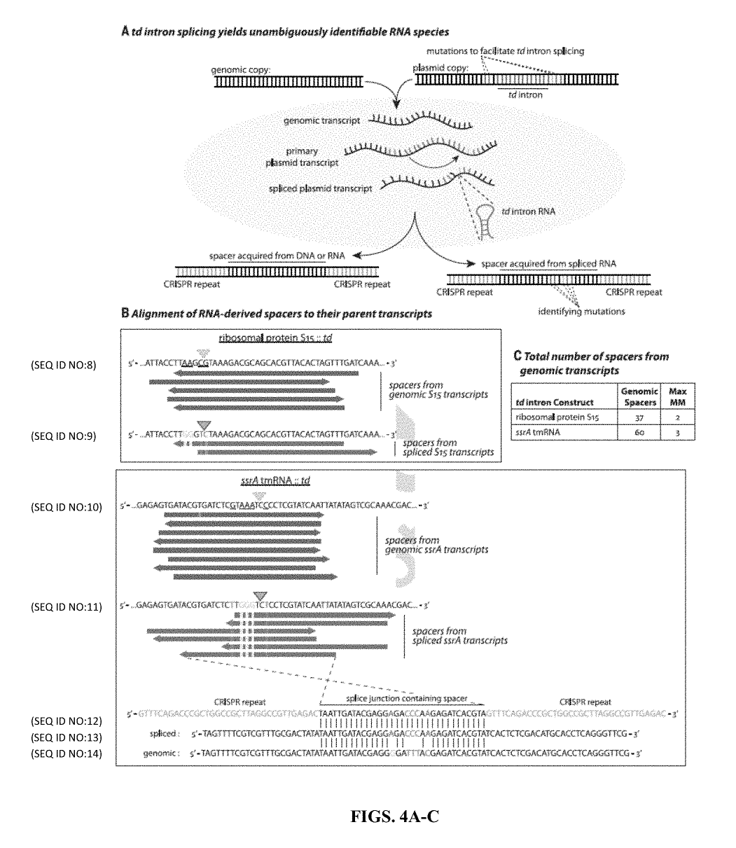

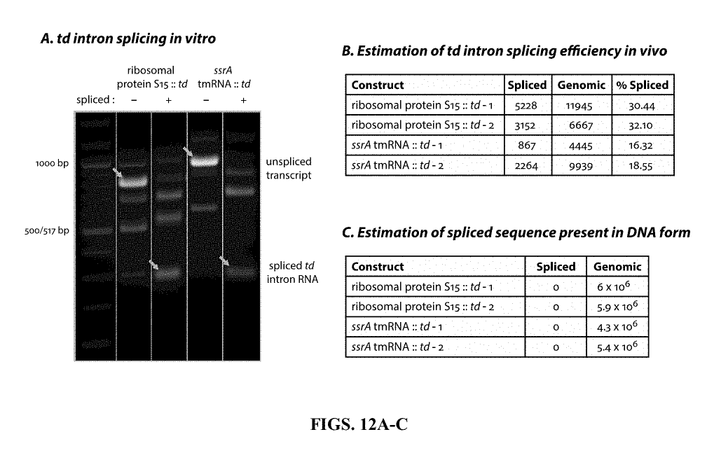

The acquisition of DNA spacer sequences from an RNA molecule can be tested by placing a functional intron into a transcript, which is spliced to yield a ligated-exon junction sequence that is then captured as DNA (Boeke et al., 1995). To test whether the RT-Cas1 complex could acquire spacers directly from RNA, the self-splicing td group I intron, a ribozyme that catalyzes its own excision from its parent transcript, was used leaving behind a splice junction that was not present as a DNA sequence (Belfort et al., 1987). Intron-interrupted versions of two MMB-1 genes—the ssrA gene, encoding a small noncoding RNA [transfer mRNA (tmRNA) (Moore and Sauer, 2007)] and Marme_0982, encoding ribosomal protein S15—in both cases inserting the intron at sites that were well sampled in the spacer libraries. Each construct was designed with four to five mutations to optimize the flanking exon sequences for td intron splicing. These mutations allowed for unambiguously distinguishing between spliced (plasmid-expressed) and native (genomic) ssrA and ribosomal protein S15 transcripts (FIG. 4A). After confirming self-splicing in vitro (FIG. 12A), the td intron-containing genes were placed on the RT-Cas1 overexpression plasmids and expressed them in MMB-1 from their native promoters. To assess the transcription level of the engineered coding regions relative to their endogenous counterparts in vivo, high-throughput sequencing of RT-PCR amplicons was performed spanning the splice junctions. It was found that ˜30% of all ribosomal protein S15 transcripts and ˜16% of all ssrA tmRNA transcripts were produced by splicing in the respective transconjugants (FIG. 12B).

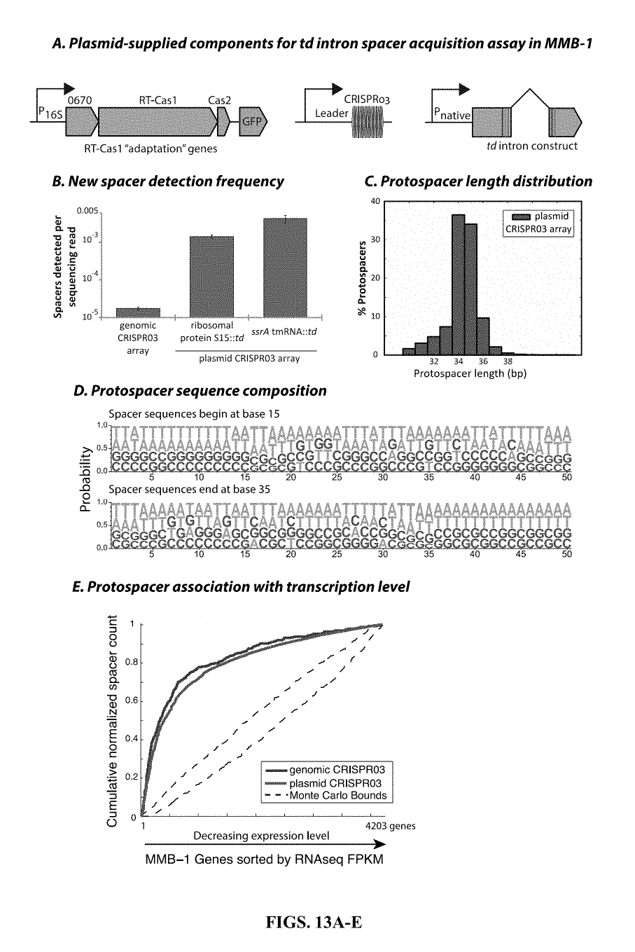

Newly integrated spacers were assayed for in plasmid copies of CRISP03, recovering 80,136 new spacers that map to the MMB-1 genome. The protospacer length, sequence composition, and bias for highly expressed genes remained consistent with the previous results in MMB-1 (FIG. 13). Two spacers were found spanning the splice junction of ribosomal protein S15 and six spacers spanning the splice junction of tmRNA from two independent cultures of two independent transconjugants, thereby confirming that the RT-Cas1 spacer acquisition machinery is capable of acquiring spacers from RNA molecules (FIGS. 4B and 4C). Both sense and antisense spacers were observed spanning the synthetic splice junctions from both the ssrA and ribosomal protein S15 constructs (FIG. 4B), further indicating flexibility in the orientation of spacer acquisition relative to the leader. The possibility that these spacers might have been acquired from an extended cDNA copy of the spliced transcripts that was generated through indiscriminate RT activity was considered. Such cDNA sequences would have been detectable by highly sensitive targeted sequencing assays and were not observed (FIG. 12C). Whereas these experiments demonstrated the ability of this system to acquire spacers from RNA, the RT-domain deletion experiments in which spacer acquisition was not biased toward transcribed regions (FIG. 3E) indicated that the system can also acquire spacers from DNA. Nonetheless, the strong transcriptional bias observed with wildtype RT-Cas1 in MMB-1 indicates that most spacer acquisitions driven by the intact RT-Cas1 fusion protein under our conditions are from RNA.

### Example 5

### Ligation of RNA and DNA oligonucleotides Directly into CRISPR Repeats by a RT-Cas1-Cas2 Complex

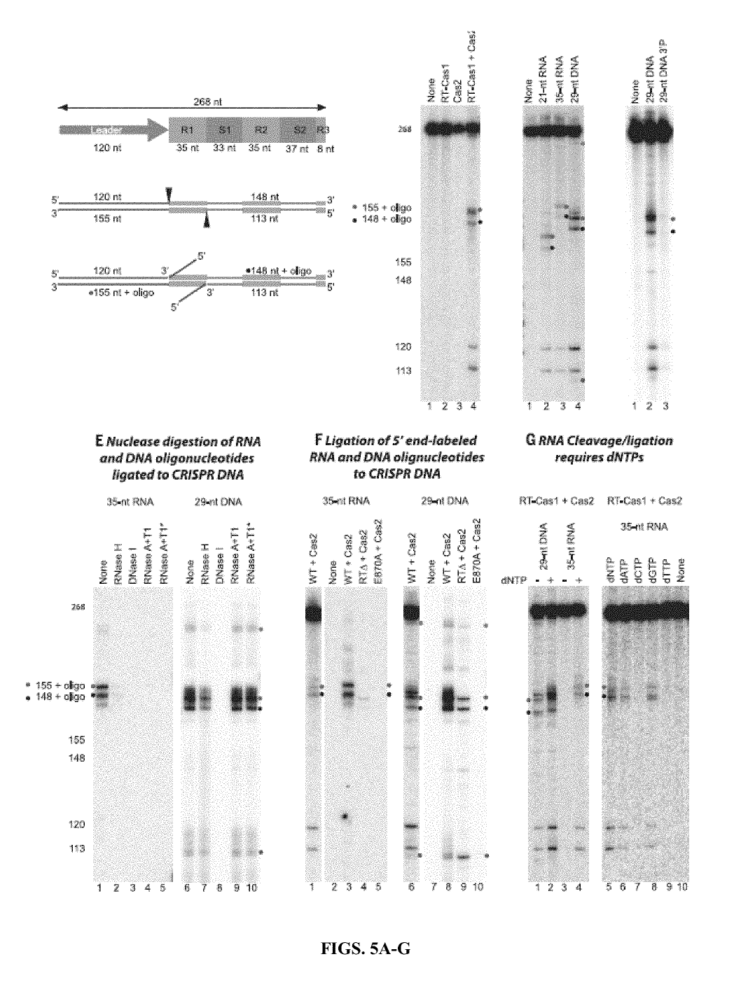

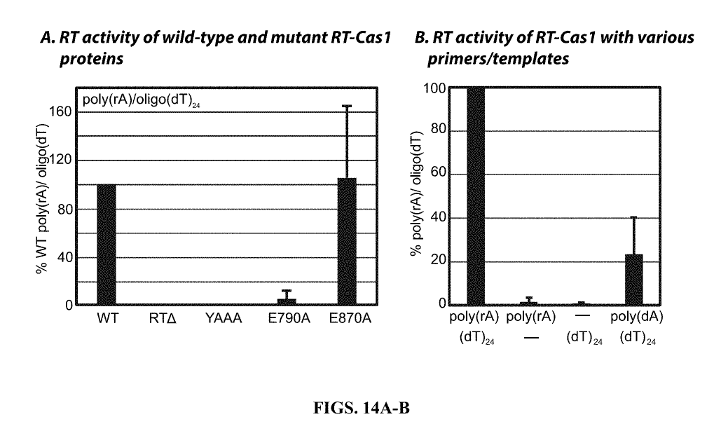

The E. coli Cas1-Cas2 complex has been shown to ligate double-stranded DNA (dsDNA) directly into a supercoiled plasmid containing a CRISPR array by means of a concerted cleavage-ligation (transesterification) mechanism, analogous to that of retroviral integrases (Nunez et al., 2015). To investigate how MMB-1 RT-Cas1 functions in spacer acquisition, this activity was reconstituted in vitro using purified RT-Cas1 and Cas2 proteins. It was confirmed that wild-type RT-Cas1 protein has RT activity that is abolished by the deletion of the RT domain (RtΔ) or mutations at the RT active site (YADD to YAAA at amino acid positions 530 to 533) (FIG. 14). To assay spacer acquisition, the purified RT-Cas1 and Cas2 proteins were incubated with (i) putative spacer precursors (protospacers) corresponding to DNA or RNA oligonucleotides of different lengths and (ii) a linear 268-bp internally labeled CRISPR DNA substrate containing the leader, the first two repeats, and interspersed spacer sequences from the MMB-1 CRISP03 array (FIG. 5A). The reactions also included deoxynucleotide triphosphates (dNTPs) to enable reverse transcription of a ligated RNA oligonucleotide.

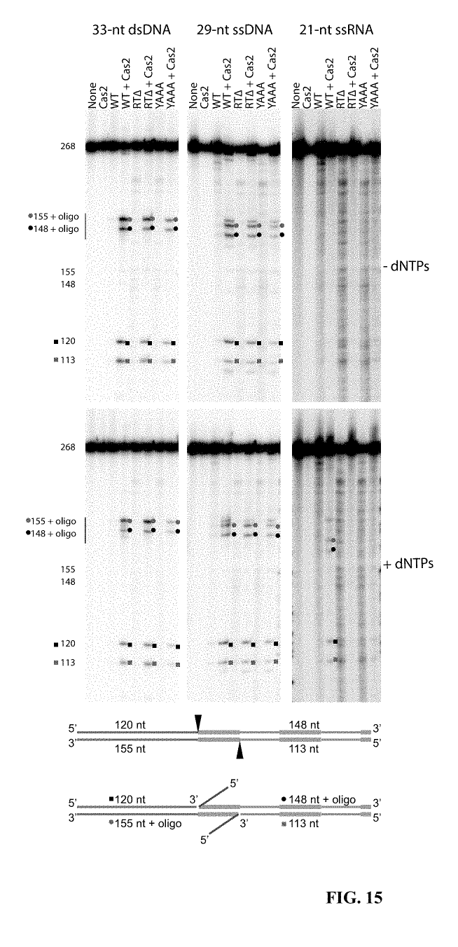

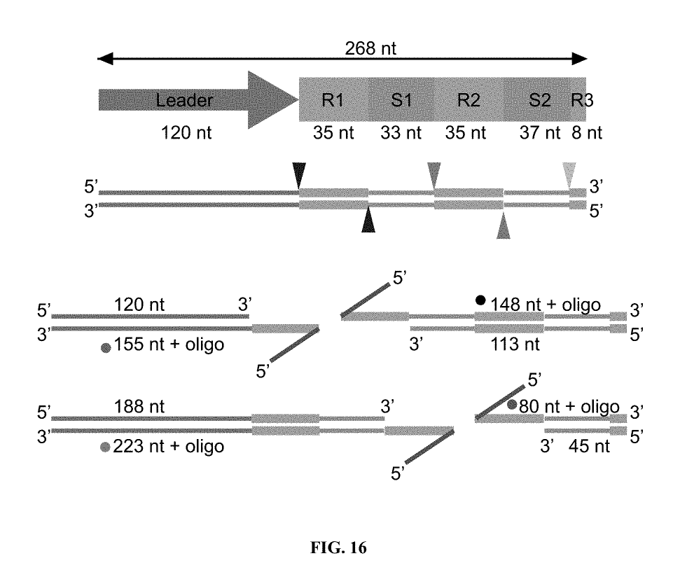

In initial assays using a dsDNA oligonucleotide, products derived from cleavage of the CRISPR substrate were readily detected in the presence of RT-Cas1 and Cas2 together but not in the presence of either protein alone (FIG. 5B). The sizes of these products were consistent with cleavage at the junctions between the leader and first repeat on the top strand and between the first repeat and spacer on the bottom strand, as expected for staggered cuts that are known to occur in type I CRISPR systems (Datsenko et al., 2012). Structural features at the leader-repeat boundary might dictate cleavage at these sites (Nuñez et al., 2015). Bands of the sizes expected for free 3′ fragments [148 and 155 nucleotides (nt)] were much weaker than those for the corresponding 5′ fragments (120 and 113 nt), reflecting their replacement with prominent bands of the sizes expected for ligation of the oligonucleotide to their 5′ ends (148 and 155 nt plus oligonucelotide). Similar products were also detected using single-stranded DNA (ssDNA) and RNA oligonucleotides of various sizes (ssDNA, 19 to 59 nt; RNA, 21 to 50 nt) (FIGS. 5B, 5C, 15, and 16), presumably reflecting that the more uniform spacer size of 34 to 36 bp in vivo is due to processing of the spacers prior to their integration into the CRISPR array. Additionally, a 3′-phosphate modification of the ssDNA oligonucleotide almost completely abolished the cleavage-ligation reaction, suggesting a crucial role of the 3′OH of the donor oligonucleotide in the integration reaction (FIG. 5D). The ligation of both DNA and RNA oligonucleotides into the CRISPR DNA was confirmed by their expected ribonuclease (RNase) and/or deoxyribonuclease (DNase) sensitivity in reactions with 5′-end-labeled oligonucleotides and unlabeled CRISPR DNA (FIG. 5E). The ligated RNA oligonucleotide was sensitive to RNase H, indicating its presence in an RNA-DNA hybrid, as would be expected if it was used as a template for cDNA synthesis by RT-Cas1 (FIG. 5E).

Although the MMB-1 RT-Cas1-Cas2 complex functions similarly to the E. coli Cas1-Cas2 complex to site-specifically integrate putative spacer precursors into CRISPR arrays, it differs in being able to use a linear CRISPR DNA substrate and to insert not only dsDNA but also ssDNA and RNA oligonucleotides. The ligation of RNA and DNA oligonucleotides into the CRISPR DNA substrate differs in two respects. First, whereas the E870A mutation at the Cas1 active site abolishes ligation of both RNA and DNA oligonucleotides, deletion of the RT domain (RtΔ) abolishes ligation of RNA but not DNA oligonucleotides (FIG. 5F). These findings mirror in vivo results showing that the E870 mutation abolishes the acquisition of both RNA and DNA spacers, whereas the RtΔ mutation abolishes the acquisition of RNA but not DNA spacers (FIGS. 3B and 3E). Second, dNTPs are required for ligation of RNA but not DNA oligonucleotides, with deoxyguanosine triphosphate (dGTP) or deoxyadenosine triphosphate (dATP) alone sufficient to support RNA ligation (FIG. 5G). Together, these findings suggest that the RT-Cas1 protein is modular, with the Cas1 domain catalyzing ligation of both RNA and DNA spacers into CRISPR repeats, but with ligation of RNA spacers requiring binding by the N-terminal and/or RT domains, possibly coupled to RT domain core closure and/or the initiation of reverse transcription on addition of dNTPs.

### Example 6

### Integrated RNA oligonucleotides are Reverse-Transcribed by the RT-Cas1-Cas2 Complex

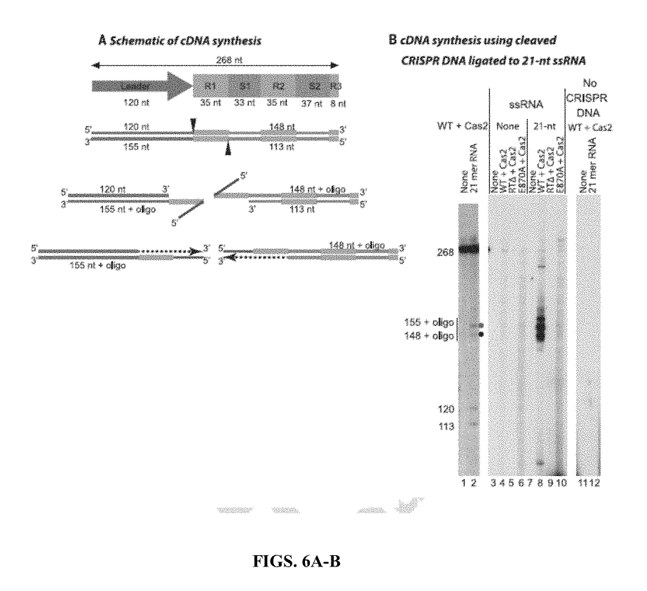

It was next tested whether the RT-Cas1-Cas2 complex could reverse-transcribe an integrated RNA oligonucleotide in vitro to generate the cDNA precursor of a fully integrated RNA spacer. The cleavage ligation reactions on either side of repeat R1 generate products with 5′ overhangs that could potentially be substrates for target DNA-primed reverse transcription (TPRT) reactions, in which the 3′ end of the opposite strand is extended to yield a DNA copy of the repeat plus the ligated RNA oligonucleotide (FIG. 6A). To detect the synthesis of such cDNAs, the CRISPR DNA was incubated with RT-Cas1-Cas2 in the presence of a 21-nt RNA oligonucleotide and supplied radioactive deoxycytidine triphosphate (dCTP) and other unlabeled dNTPs during the incubation (FIG. 6A). cDNA synthesis during the reactions was evident by the labeled products being of the same size as the two ligation products, as expected for a TPRT reaction extending through the R1 repeat and ligated RNA.

The synthesis of these cDNAs depends on the presence of the RNA oligonucleotide, the CRISPR DNA, and RT-Cas1-Cas2 (FIG. 6B). The RtΔ mutant abolishes cDNA synthesis, whereas the E870A mutant, which retains RT activity (FIG. 14) but cannot integrate the RNA oligonucleotide or create the 3′OH required for priming cDNA synthesis (FIG. 5F), produces only a heterogeneous background of labeled products (FIG. 6B). The TPRT products detected in the assays may represent an intermediate in spacer acquisition, with additional steps potentially including digestion of the ligated RNA spacer strand by a host RNase H, synthesis of a fully dsDNA containing the spacer sequence by RT-Cas1 or a host DNA polymerase, and ligation of the unattached ends of the dsDNA into the CRISPR array. The in vivo and in vitro data suggest that this can occur in either orientation and may involve host enzymes that are present in MMB-1 but not in E. coli.

It was then shown that the MMB1 RT-Cas1 fusion protein can mediate the direct acquisition of spacers from donor RNA, using the Cas1 integrase activity to directly ligate an RNA protospacer into CRISPR DNA repeats. The 3′ end generated by cleavage of the opposite DNA strand is then poised for use as a primer for TPRT (Zimmerly et al., 1995). This mechanism shares features with group II intron retrohoming, in which the intron RNA uses its ribozyme activity to insert itself directly into the host genome and is then converted to an intron cDNA by using the 3′ end generated by cleavage of the opposite DNA strand for TPRT (Lambowitz and Zimmerly, 2004). Because type III CRISPR systems are known to target RNA for degradation, and RT-Cas1-encoding genes are exclusively associated with such systems, RNA spacer acquisition makes these CRISPRs uniquely capable of generating immunity against parasitic RNA sequences, potentially including RNA phages and/or other “selfish” RNAs that maintain themselves through the action of host machinery (Blumenthal and Carmichael, 1979; Biebricher and Orgel, 1973; Konarska and Sharp, 1989; Flores et al., 2014). The acquisition of RNA spacers might also contribute to immune responses to highly transcribed regions of DNA phages and plasmids. This Cas1 could then be coupled to an interference system that targets DNA, RNA, or both (Marraffini and Sontheimer, 2008; Hale et al., 2009; Hale et al., 2012; Tamulaitis et al., 2014; Goldberg et al., 2014; Peng et al., 2015; Samai et al., 2015).

It is possible that fusion between the RT and Cas1 domains may not be necessary to facilitate uptake of RNA spacers; there are several examples of CRISPR loci in which genes encoding similar group II intron-like RTs are adjacent but not fused to Cas1 (Simon and Zimmerly, 2008). Thus, the mechanisms described in the present disclosure could potentially extend to species with separately encoded RT and Cas1 components. In addition, RNA spacer acquisition could be involved in gene regulation, providing a straightforward means for bacteria to down-regulate a set of target loci in response to activation of the CRISPR locus.

To fully assess the prevalence and importance of CRISPR adaptation to RNA, a greater understanding of the impact of invasive RNAs in bacteria is necessary. However, the knowledge of the abundance and distribution of RNA phages and other RNA parasites is limited, with the vast majority restricted to the Escherichia and Pseudomonas genera. Future research on the distribution of spacers in RT-associated CRISPR loci among natural populations of bacteria and their environments might help shed light on this topic.

### Example 7

### Materials and Methods

RT-Cas1 genomic neighborhood analysis: The genomic neighborhoods (up to 20 kb) of RTCas1-encoding genes were retrieved from 50 bacterial strains with a custom BioPython script that uses the NCBI tblastn software. The HMMER 3.0 algorithm was then used to identify whether the RT-Cas1-encoding genes were associated with type I, II, or III CRISPR systems, using Cas3 (TIGR 01587, 01596, 02562, 02621, and 03158), Cas9 (TIGR 01865 and 3031), and Cas10 (TIGR 02577 and 02578) hidden Markov models as “signature” genes for each type, respectively (Makarova et al., 2011). Each result was assessed manually by iterative runs of BLAST (Basic Local Alignment Iterative Search Tool, NCBI) and the CRISPR finder online suite.

Monte Carlo simulation of expected spacer acquisition characteristics for random sampling of all genes: A Monte Carlo simulation was used to evaluate a null hypothesis based on a random assortment of spacer acquisitions from genomic DNA, with no dependence on gene expression level. For each system, a series of samples of 500 spacers each were randomly chosen in silico from a list of all genes, based on the sizes of the individual genes using the stochastic universal sampling algorithm. Sets of 1000 such trials were used to generate a range of null relationships between gene expression and spacer acquisition. The Monte Carlo bounds depict the envelope of such simulated random assortments. Traces above this envelope indicate preferential spacer acquisition from highly expressed genes; traces below the envelope indicate spacer acquisition from poorly expressed genes more often than expected by random chance. RNAseq data from the E. coli K12 genome were obtained from (Haas et al., 2012) (data set without computational background subtraction). MMB-1 expression data were generated by RNAseq analysis of the transconjugants used in this study (FIG. 3).

Construction of expression vectors: Plasmids for inducible overexpression of the MMB-1 type III-B CRISPR operon in E. coli were built on the pBAD/Myc-His B backbone (Life Technologies). RT-Cas1-associated genes [Marme_0670, Marme_0669 (RT-Cas1), and Marme_0668 (Cas2)] and green fluorescent protein (GFP) were driven by Para, and the CRISP03 array was driven by Ptrc. The other seven genes [Marme_0677 to 0672 (Cmr1 to -6) and Marme_0671] and lacZα were driven by Plac. GFP and lacZα ORFs enabled verification of expression of the transcripts containing RT-Cas1-associated adaptation genes and Cmr effector genes, respectively. Point mutants of the Cas1 (E790A or E870A) and RT domains (YADD to YAAA at amino acid positions 530 to 533) of the RT-Cas1-encoding gene were tested with overexpression of the RT-Cas1-associated subset, with and without the remaining seven genes. Deletion mutants of the RT domain of RT-Cas1 (Δ299-588), and Cas2 (Δ32-92) were tested with overexpression of the RTCas1-associated subset only.

Plasmids for the overexpression of the RTCas1-associated genes in MMB-1 cells were built on the pKT230 backbone (a gift from L. Banta, Williams College). The genes were driven by the 100-bp promoter-containing sequence (MMB-1 chromosome position 306879 to 306978) upstream of a MMB-1 16S rRNA gene. Cas1 point mutants (E790A or E870A) and the RTΔ mutant were also tested. For experiments with td intron-containing constructs, a copy of the CRISP03 array with its leader sequence was also placed on the pKT230 vector to increase the concentration of CRISPR arrays per unit input DNA in the PCR amplification step, and thus increase the efficiency of the spacer detection assay.

Plasmids for protein expression and purification were built on the pMal-c2X backbone [New England Biolabs (NEB)] for RT-Cas1 (wild type and mutants) and on the pET14b backbone (Novagene) for Cas2. Variants of RT-Cas1 were expressed with an N-terminal maltose-binding protein tag attached via a noncleavable rigid linker (Mohr et al., 2013). Cas2 was expressed with a N-terminal 6xHis tag. All plasmids were verified by sequencing.

Strains and culture conditions: All bacterial strains used in this study were stored in 20% glycerol at -80° C. Two clones from each conjugation were maintained for each plasmid (referred to as independent transconjugants).

pBAD plasmids (AmpR) encoding MMB-1 type III-B operon components were transformed into chemically competent TOP10F′ cells (Life Technologies). TOP10F′-derived strains were grown at 37° C. on Luria-Bertani (LB) agar plates (10 g/l tryptone, 5 g/l yeast extract, 10 g/l NaCl, and 15 g/l agar) with 100 mg/ml of ampicillin, 0.1% w/v arabinose, and 0.1 mM IPTG (isopropyl-β-D-thiogalactopyranoside) overnight.

pKT230 plasmids (KanR) encodingMMB-1 type III-B operon components were mobilized into a spontaneous rifampicin-resistant mutant of MMB-1 (strain ATCC 700492) from a donor E. coli strain carrying the pRL443 conjugal plasmid (a gift from M. Davison, Carnegie Institution), as described in (51). All transformed MMB-1 strains were grown on 2216 marine agar (Difco) with 50 mg/ml of kanamycin for 16 hours at 25° C.

For experiments with MMB-1 transconjugants carrying td intron constructs, 150-ml cultures were subsequently prepared in 2216 broth (Difco) with 50 mg/ml of kanamycin and shaken at 26° to 27° C. in 1-liter flasks for 20 hours before midiprep. E. coli strain DH5a (Life Technologies) was used for cloning and Rosetta2 and Rosetta2 (DE3) (Novagen) were used for protein expression. Bacteria were grown in LB medium with shaking at 200 rpm. Antibiotics were added when needed (ampicillin, 100 mg/1; chloramphenicol, 25 mg/l).

Nucleic acid extraction: Plasmid DNA from E. coli strains was extracted using the QIAprep Spin Miniprep Kit (QIAGEN). Genomic DNA fromMMB-1 strains was extracted using a modified SDS-protease K method: Briefly, cells were scraped from plates and resuspended in 1 ml of lysis buffer (10 mMtris, 10 mM EDTA, 400 mg/ml proteinase K, and 0.5% SDS) and incubated at 55° C. for 1 hour. Digest (50 to 100 ml) was subsequently purified using the Genomic DNA Clean & Concentrator Kit (Zymo Research).

Total RNA was extracted from MMB-1 strains using a combined trizol-RNeasy method: Briefly, cells were scraped from plates and homogenized directly in 1 ml of trizol (Life Technologies) by vortexing, and total RNA was extracted with 200 ml of chloroform. Ethanol (500 ml) was added to an equal volume of the aqueous phase containing RNA, and the mixture was purified using the RNeasy Kit (QIAGEN) with on-column DNase digestion according to the manufacturer's instructions. This protocol selects RNA >200 nt and thus depletes transfer RNAs. Plasmid DNA was purified from large MMB-1 cultures using a custom midi prep method. Cells were harvested from 150- to 200-ml confluent cultures (3000 g, 30 min, 4° C.) and homogenized in 12 ml of alkaline lysis buffer (40 mM glucose, 10 mM tris, 4 mM EDTA, 0.1 N NaOH, and 0.5% SDS) at 37° C. by pipetting until clear (10 to 15 min). Chilled neutralization buffer (8 ml) was added (3 M CH3COOK and 2 M CH3COOH), and lysates were immediately transferred to ice to prevent digestion of genomic DNA. Samples were mixed by inverting, and the genomic DNA-containing precipitate was removed by centrifugation (20,000 g, 20 min, 4° C.). Clarified lysates were extracted twice with a 1:1 mixture of tris-saturated phenol (Life Technologies) and CHCl3 (Fisher Scientific) and once with CHCl3 in heavy phase lock gel tubes (5 Prime). Ethanol (50 ml) was added and DNA was pelleted by centrifugation (16,000 g, 20 min, 4° C.), washed twice in 80% ethanol, and resuspended in 500 μof elution buffer (10 mM tris, pH 8.5). Samples were treated with 20 μg/ml RNase A (Life Technologies) at 37° C. for 30 min, further digested with 150 μg/ml of proteinase K in 0.5% SDS at 50° C. for 30 min, and purified by organic extraction. Plasmid DNA was resuspended in 0.5 ml of elution buffer, desalted with Illustra NAP-5 G-25 Sephadex columns (GE Healthcare), and eluted with 1 ml of water. Batches of 100 μl were linearized with PvuII-HF (NEB) to aid denaturation during PCR. Last, each digest was purified using a Genomic DNA Clean & Concentrator column (Zymo Research). DNA and RNA preparations were quantified using a fluorometer (Qubit 2.0, Life Technologies).

Spacer Sequencing: Leader proximal spacers were amplified by PCR from 3 to 4 ng of genomic DNA per ml of PCRmix using

forward primer AF-SS-119
(CGACGCTCTTCCGATCTNNNNNCTGAAATGATTGGAAAAAATAAGG,
SEQ ID NO: 15)

anchored in the leader sequence and

reverse primer AF-SS-121
(ACTGACGCTAGTGCATCACGTGGCGGAGATCTTTAA, SEQ ID
NO: 16)

in the first native spacer. For each sample, 96 10-μl reactions were pooled. Sequencing adaptors were then attached in a second round of PCR with 0.01 volumes of the previous reaction as a template, using

AF-SS-44:55
(CAAGCAGAAGACGGCATACGAGATNNNNNNNN
GTGACTGGAGTTCAGACGTGTGCTCTTCCGATCACTGACGCTAGTGCAT
CA, SEQ ID NO: 17)
and
AFKLA-67:74
(AATGATACGGCGACCACCGAGATCTACAC NNNNNNNN
ACACTCTTTCCCTACACGACGCTCTTCCGATCT, SEQ ID NO: 18),

where the (N)8 barcodes correspond to TruSeq HT indexes D701 to D712 (reverse-complemented) and D501 to D508, respectively (Illumina). Template matching regions in primers are underlined. Phusion High-Fidelity PCR Master Mix with HF Buffer (Fisher Scientific) was used for all reactions. Cycling conditions for round 1 were as follows: one cycle at 98° C. for 1 min; two cycles at 98° C. for 10 s, 50° C. for 20 s, and 72° C. for 30 s); 24 cycles at 98° C. for 15 s, 65° C. for 15 s; and 72° C. for 30 s); and one cycle at 72° C. for 9 min. Conditions for round 2 were one cycle at 98° C. for 1 min; two cycles at 98° C. for 10 s, 54° C. for 20 s, and 72° C. for 30 s; five cycles at 98° C. for 15 s, 70° C. for 15 s, and72° C., 30 s; and one cycle at 72° C. for 9 min. The dominant amplicons containing the first native spacer from unmodified CRISPR templates after rounds 1 and 2 were 123 bp and 241 bp, respectively. We prepared sequencing libraries by blind excision of gel slices at 300 to 320 bp (70 bp above the 241-bp band, consistent with the expected size of an amplicon from an expanded CRISPR array) after agarose electrophoresis (3%, 4.2 V/cm, 2 hours) of the round 2 amplicons.

When amplifying spacers from plasmids, 1 ng of DNA was used per microliter of PCR mix, synthesis time was shortened to 15 s, and 20 and nine cycles were used in rounds 1 and 2 instead of 24 and five, respectively. Additionally, round 1 amplicons were purified by blind excision of gel slices at 180 to 200 nt after denaturing PAGE (polyacrylamide gel electrophoresis) [pre-run TBEUrea 10% gels (Novex), 180 V, 80 min in XCell SureLock Mini-Cells (Life Technologies)], and agarose gel-purified libraries were further PAGEpurified by blind excision of gel slices at 300 to 320 nt (pre-run TBE-Urea 6% gels, 180 V, 90 min as above). In this way, spacer detection efficiency was increased ˜100-fold. Libraries were quantified by Qubit and sequenced with MiSeq v3 kits (Illumina) (150 cycles, read 1; 8 cycles, index 1; and 8 cycles, index 2).

Spacers were trimmed from reads using a custom Python script and considered identical if they differed only by one nucleotide. Protospacers were mapped using Bowtie 2.0 (“very-sensitive local” alignments). These methods preserve strand information.

Directional RNAseq profiling of MMB-1 strains: Total RNA (1 μg) was incubated at 95° C. in alkaline fragmentation buffer (2 mM EDTA, 10 mM Na₂CO₃, and 90 mM NaHCO₃; pH-9.3) for 45 min and PAGE-purified [pre-run 15% TBE-Urea precast gels, 200 V, 45 min in Mini-PROTEAN electrophoresis cells (Bio-Rad)] to select 30- to 80-nt fragments. RNA fragments were 3′ -dephosphorylated with T4 polynucleotide kinase (NEB) at 37° C. for 60 min in the supplied buffer, then desalted by ethanol precipitation. Desphosphorylated RNA was denatured again in adenylated ligation buffer [3.3 mM dithiothreitol (DTT), 10 mM MgCl₂, 10 μg/ml acetylated BSA, 8.3% glycerol, and 50 mM HEPES-KOH; pH ˜8.3) for 1 min at 98° C. and ligated to pre-adenylated adaptor AF-JA-34 (/5rApp AGATCGGAAGAGCACACGTCT/3ddC/, SEQ ID NO: 19) at 22° C. for 4 hours using 10 U T4 RNA Ligase I (NEB). The (N)₆ barcode for each RNA fragment allowed us to computationally collapse PCR bias. Excess adaptor was removed by treatment with 5′ deadenylase (NEB) followed by RecJf (NEB) treatment and organic extraction to purify ligation products. RNA was reverse transcribed using primer AF-JA-126 (/5Phos/AGATCGGAAGAGCGTCGTGT/iSp18/CACTCA/iSp18/GTGACTGGAGTTCAGACGTGTGCTCTTCCGATCT, SEQ ID NO: 20) with SuperScript II (Life Technologies) and subsequently hydrolyzed in 0.1 M NaOH at 70° C. for 15 min. cDNAwas PAGE-purified (pre-run 10% TBE-urea gels, 200 V, 45 min in Mini-PROTEAN electrophoresis cells) to select 90- to 150-nt fragments and circularized with 50U CircLigase I (Epicentre). Libraries were prepared by six to 14 cycles of PCR with universal adaptor AF-JA-158 (AATGATACGGCGACCACCGAGATCTACACTCTTTCCCTACACGACGCTCTTCCGATC T, SEQ ID NO: 21) and indexing primers AF-JA-118:125 (CAAGCAGAAGACGGCATACGAGAT NNNNNN GTGACTGGAGTTCAGACGTGTGCTCTTCCG, SEQ ID NO: 22) where the (N)₆ barcodes correspond to TruSeq LT indexes AD001 to AD008 (Illumina). Amplicons of 160 to 200 bp were gel purified by agarose electrophoresis.

Construction and validation of td intron constructs: Constructs with the following features were ordered as gBlocks (Integrated DNA Technologies) and cloned downstream of the T7 promoter in pCR-Blunt II-TOPO (Life Technologies). Bases 208 to 216 (CTTAAGCGT) of the ribosomal protein S15 gene (Marme_0982) and bases 67 to 75 (CGTAAATCC) of the ssrA tmRNA gene (Marme_R0008) were replaced with the wild-type td intron splice junction (CTTGGGT|CT). The 393-bp intron sequence was inserted at the exon junction|. Included were 128 bp of upstream sequence for Marme_0982 and 183 bp of upstream sequence and30bp of downstream sequence for Marme_R0008. Transcripts were generated from linearized plasmids using the MEGAscript T7 Transcription kit (Life Technologies). Mostly unspliced RNA was obtained by arresting the transcription reaction after 5 min at 37° C. and subsequently extracting it with acidified phenol:CHCl3 (Life Technologies). One-third of the reaction product was incubated in a splicing buffer (40 mM tris at pH 7.5, 6 mM MgCl₂, 100 mM KCl, and 1 mM ribo-GTP) at 37° C. for 30 min and desalted by ethanol precipitation. Spliced and unspliced transcripts were visualized by 1/4× tris-acetate-EDTA native agarose gel electrophoresis, with a 100-bp Quickload dsDNA ladder (NEB) providing approximate sizing. Intron containing genes were then transferred to pKT230-derived MMB-1 overexpression vectors carrying RT-Cas1-associated genes and a copy of the CRISP03 array. One clone each from two independent conjugations was isolated for each vector.

In vivo splicing efficiency was measured by high-throughput sequencing as follows. Total RNA was extracted and 1 μg was reverse-transcribed (SuperScript III, high GC content protocol; Life Technologies) with gene-specific primers downstream of the splice junctions that would bind both spliced and unspliced transcripts: AF-SS-238 (CTTAGCGACGTAGACCTAGTTTTT, SEQ ID NO: 23) for Marme_0982 and AF-SS-241 (GGTTATTAAGCTGCTAAAGCGTAG, SEQ ID NO: 24) for Marme_R0008. cDNA was treated with RNase H, and libraries were prepared by a two round PCR method adapted from the CRISPR spacer sequencing method described above. Round 1 of PCR was performed at annealing temperatures of 48° and 65° C. for two and 19 cycles, respectively, with primers

AF-SS-242
(CGACGCTCTTCCGATCTNNNNNGATTCGCATGGTAAAC, SEQ ID
NO: 25)
and
AF-SS-243
(ACTGACGCTAGTGCATCAAACTAGTGTAACGTGCTG, SEQ ID
NO: 26)

for Marme_0982, and for two and 16 cycles, respectively, with primers

AF-SS-247
(CGACGCTCTTCCGATCTNNNNNCACGAACCTGAGGTG, SEQ ID
NO: 27)
and
AF-SS-248
(ACTGACGCTAGTGCATCACGTCGTTTGCGACTATATAATTGA, SEQ
ID NO: 28)

for Marme_R0008. This approach simultaneously generated amplicons of identical length for both spliced and unspliced transcripts, which were then attached to adaptors (Illumina) with a second round of PCR as before.

The presence of exon-junction sequences corresponding to the td intron constructs in DNA form outside the CRISPR arrays was also tested by high-throughput sequencing. Libraries consisting of the ˜100-bp region containing the td intron insertion sites in Marme_{—R}0008 and Marme_0982 were prepared by a two-round PCR method identical to the one described above for measuring splicing efficiency by RT-PCR, using 100 ng of genomic DNA (˜2×107 copies) as a template instead of reverse-transcribed cDNA. Round 1 of PCR was performed at annealing temperatures of 57° C. and 68° C. for two and 16 cycles, respectively, with primers

AF-SS-318
(CGACGCTCTTCCGATCTNNNNNCACATTCATGACCACCATTCTCG,
SEQ ID NO: 29)
and
AF-SS-309
(ACTGACGCTAGTGCATCACTTCGGTCTTAGCGACGTAGAC,
SEQ ID NO: 30)

for Marme_0982 and primers

AF-SS-310
(CGACGCTCTTCCGATCTNNNNNGGGGTGACATGGTTTCGACG, SEQ
ID NO: 31)
and
AF-SS-311
(ACTGACGCTAGTGCATCAGCAGGTTATTAAGCTGCTAAAGCG, SEQ
ID NO: 32)

for Marme R0008. The amplicons were then attached to adaptors (Illumina) with a second round of PCR as before. Each library was sequenced to a depth of ˜5million reads. To ensure that the PCR was not bottlenecked, we also included a spike-in (1 molecule per 1000 copies of the MMB-1genome) of synthetic ssDNA templates-AF-SS-312 (TAAAAACATTGAAGGTCTA CAAGGTCACTTTAAAGCTCACATTCATGACCACCATTCTCGTCGCNNNNNNNNNNNN ATGGTAAACCAACGTCGTAAGTTGTTGGATTACCAGCTGCGTAAAGACGCAGCACG TTACACTAGTTTGANNNNNNNNNNNNGTCTACGTCGCTAAGACCGAAG, SEQ ID NO: 33) for Marme_0982 and AF-SS-313 (GGGGTGACATGGTTTCGACG NNNNNNNNNNNNCCTGAGGTGCATGTCGAGAGTGATACGTGATCTCAGCTGTCCCC TCGTATCAATTATATAGTCGCAAANNNNNNNNNNNNCGCTTTAGCAGCTTAATAAC CTGCTAGTGTGCTGCCCTCAGGTTGCTTGTAGCCCGAGATTCCGCAGT, SEQ ID NO: 34) for Marme_{—R}0008—that could be amplified concomitantly by the same primer sets to yield identically sized amplicons.

The spike-in derived reads are easily identified by sequence, with the diversity of randomized (N)₁₂ segments used to evaluate the degree to which distinct reads in the amplified pool represent independent molecules from the pre-amplification mixture. A large number of spike-in barcodes (ideally a different barcode for every spike-in read) indicate that a high fraction of reads from the amplified pool represent unique molecules in the initial sample, whereas repeated appearances of a small number of (N)₁₂ barcodes in the amplified pool would be indicative of bottleneck formation during PCR (and hence a less than optimal relationship between read counts and molecules in the initial pool). For the purpose of estimating the number of molecules sampled from an initial pool, we calculated a nonredundancy fraction, which is the ratio of spike-in-derived barcodes to total spike-in-derived reads. The nonredundancy fraction provides a multiplier that can be used to correct raw read counts from an amplified pool to obtain an estimate of the contributing number of molecules from the initial pool. This is particularly applicable for estimating a minimal incidence of a rare class (i.e., setting a detection limit for spliced copies of the td intron-containing DNA constructs in this work). Given nonredundancy fractions of >0.45 for all samples in these experiments, the observed totals of control (nonspliced, genomic) sequence reads (FIG. 12C) would have been sufficient to detect the presence of extended spliced td intron-containing DNA molecules, even at the low incidence of 10⁻⁶. The same cultures of MMB-1 were used to assess both splicing efficiency and the presence of exon-junction sequences in DNA form.

PCR Fidelity: Analyzing sequence distributions through PCR and sequencing entails certain best practices in terms of both experimental protocols and analysis. In particular, several precautions were observed in constructing sequencing libraries for spacer sequencing. PCR titrations were performed to ensure that the amplification kinetics were in the linear range of the reactions before any size selection step (e.g., band excision from native agarose gels); this avoids renaturation artifacts in complex sequence pools. The overall error rate was empirically determined for every experiment by analyzing the distribution of mismatches in the sequences obtained from the first native spacer in the CRISP03 array; this enabled the estimation of the error rate in the region of the sequencing reads that contained newly acquired spacers. PCR bottlenecking was also measured as the number of repeat occurrences of any given new spacer. All synthetic sequences that could lead to confounding contamination issues were avoided: No sequences from E. coli , MMB-1, or other sources have been synthesized as amplifiable substrates. As a benchmark for recovery of individual sequences, a nonbacterial sequence was synthesized as a spacer flanked by the appropriate CRISPR repeats. This repeat-flanked spacer sequence (CTGGGACATATAATATCGTCCCCGTAGATGCCTAT (SEQ ID NO: 35); a segment of the phage MS2) was recovered effectively in experiments with an E. coli transformant carrying a plasmid with the indicated template. Appearances of MS2 sequences in other trials were limited to this single sequence, indicating a likely source due to a low level of cross sample “bleeding.”

Protein purification: Expression plasmids were transformed into E. coli strains Rosetta2 (pMal derivatives) or Rosetta2 (DE3), and single transformed colonies were grown in an LB medium supplemented with appropriate antibiotics over night at 37° C. with shaking. Six flasks each containing 1 liter LB were inoculated with 1% of the overnight culture and grown at 37° C. with shaking to log phase. After the culture reached an optical density at 600 nm of ˜0.8, IPTG was added to 1 mM final concentration and the cultures were incubated at 19° C. for 20 to 24 hours. Cells were harvested by centrifugation and the pellet was dissolved in A1 buffer (25 mM KPO4, pH 7; 500 mM NaCl; 10% glycerol; 10 mM β-mercaptoethanol; 10 ml/g cell paste) on ice. Lysozyme was added to 1 mg/ml final concentration and incubated at 4° C. for 0.5 hours. Cells were then sonicated (Branson Sonifier 450; three bursts of 15 s each with 15 s between each burst). The lysate was cleared by centrifugation (29,400 g, 25 min, 4° C.), and polyethyleneimine (PEI) was added to the supernatant in six steps on ice with stirring to a final concentration of 0.4%. After 10 min, precipitated nucleic acids were removed by centrifugation (29,400 g, 25 min, 4° C.), and proteins were precipitated from the supernatant by adding ammonium sulfate to 60% saturation on ice and incubating for 30 min. Proteins were collected by centrifugation (29,400 g, 25 min, 4° C.), dissolved in 20 ml A1 buffer, and filtered through a 0.45-mm polyethersulfone membrane (Whatman Puradisc).

Protein purification was achieved by using a BioLogic fast protein liquid chromatography system (BioRad). RT-Cas1 was purified by loading the filtered crude protein onto an amylose column (30 ml; NEB Amylose High Flow resin), washing with 50 ml of A1 buffer, followed by 30 ml A1 plus 1.5M NaCl and 30 ml of A1 buffer. Bound proteins were eluted with 50 ml of 10 mM maltose in A1 buffer. Fractions containing RT-Cas1 were identified by SDS-PAGE, pooled, and diluted to 250 mM NaCl. The protein was then loaded onto a 5-ml heparin-Sepharose column (HiTrap Heparin HP column; GE Healthcare) and eluted with a 100 mM to 1-M NaCl gradient. Peak fractions (˜700 mM NaCl) were identified by SD S-PAGE, pooled, and dialyzed into A1 buffer. The dialyzed protein was concentrated to >10 mM using an Amicon Ultra Centrifugal Filter (Ultracel-50K). The protein was stable in A1 buffer on ice for about 3 months.

The initial steps in the Cas2 purification were similar, except that the cell paste was resuspended in N1 buffer (25 mM tris-HCl, pH 7.5; 500 mM KCl; 10 mM imidazole; 10% glycerol; and 10 mM DTT) and the ammonium sulfate precipitation step was omitted. Instead, the Cas2 PEI supernatant was loaded directly onto a 5-ml nickel column (HiTrap Nickel HP column; GE Healthcare) and eluted with an imidazole gradient (60 ml 10 to 500 mM in N1 buffer). Peak fractions containing Cas2 were identified by SD S-PAGE and pooled. After adjusting the KCl concentration to 200 mM, the pooled fractions were loaded onto two tandem 5-ml heparin-Sepharose columns. The protein was eluted with a linear KCl gradient (50 ml, 100 mM to 1 M), and Cas2 peak fractions (˜800 mM KCl) were identified by SDS-PAGE and stored on ice in elution buffer. The protein was stable on ice for several months. All protein concentrations were measured using the Qubit Protein assay kit (Life Technologies) according to the manufacturer's protocol. Proteins were >80% pure based on densitometry.

Formation of RT-Cas1+Cas2 complex: Purified RTCas1 (2500 pMol) was mixed with a two-fold excess of purified Cas2 in 250 mM KCl, 250 mM NaCl, and 12.5 mM tris-HCl (pH 7.5); 12.5 mM KPO₄ (pH7); 5 mM DTT; 5 mM BME; and 10% glycerol and incubated on ice for >16 hours prior to reactions.

RT assay: RT assays with poly(rA)/oligo(dT)₂₄ were performed by pre-incubating poly(rA)/oligo(dT)₂₄ (80 μM and 50 μM, respectively) in 200 mM KCl, 50 mM NaCl, 10 mM MgCl₂, and 20 mM tris-HCl (pH 7.5); 1 mM unlabeled deoxythymidine triphosphate (dTTP); and 5 mCi [α-32P]-dTTP (3000 Ci/mmol; PerkinElmer) for 2 min at the desired temperature, then initiating the reaction by adding the RT-Cast proteins (1 to 2 mM final concentration). The reactions (20 to 30 ml) were incubated for times up to 30min. A 3-μl sample was withdrawn at each time point and added to 10 μl of stop solution (0.5% SDS and 25 mM EDTA). Reaction products were spotted onto Whatman DE81 paper (10×7.5-cm sheets; GEHealthcare Biosciences), which was then washed three times with 0.3M NaCl and 0.03 M sodiumcitrate, dried, and scanned with a Phosphorlmager (Typhoon Trio Variable Mode Imager; GEHealthcare Biosciences) to quantify the bound radioactivity.

CRISPR DNA cleavage/ligation assay: MMB-1 CRISPR DNA substrate was a PCR product amplified with primers MMB 1 cri sp5b (CACTCGACCGGAATTATCGACGAA, SEQ ID NO: 36) and MMB1crisp3 (TCTGAAACTCTGAATACTAACGAAAAATAG, SEQ ID NO: 37) using Phusion High-fidelity DNA polymerase according to the manufacturer's protocol (NEB or Thermo Scientific). The resulting 268-bp PCR fragment contains 120 bp of the leader, 35 bp of repeat 1, 33 bp of spacer 1, 35 bp of repeat 2, 37 bp of spacer 2, and 8 bp of repeat 3. Internally labeled substrate was prepared by adding 25 μCi [α-³²P]-dTTP or dCTP (Perkin Elmer) and 40 μM dTTP or dCTP, respectively, to the PCR reactions. Labeled DNA was purified by electrophoresis in a native 6% polyacrylamide gel, cutting out the labeled band, and electro-eluting the DNA using midi DTube dialyzer cartridges (Novagen). The eluted DNA was extracted with phenol:chloroform:isoamyl alcohol (phenol-CIA), ethanol-precipitated, and quantitated using a Qubit dsDNA assay kit (Life Technologies).

CRISPR DNA cleavage-ligation assays contained RTCas1 -Cas2 complex (500 nM final), MMB-1 CRISPR substrate (1 nM), 20 mM tris (pH 7.5), and 7.5 mM free MgCl2. DNA or RNA oligonucleotides and dNTPs or Mg²⁺ were added at 2.5 mM and 1 mM final concentrations as indicated for individual experiments. Reactions were incubated at 37° C. for 1 hour and stopped by adding phenol-CIA. The supernatant was mixed at a 2:1 ratio with loading dye (90% formamide, 20 mM EDTA, and 0.25 mg/ml bromophenol blue and xyan cyanol), and nucleic acids were analyzed in a 6% polyacrylamide 7 M urea gel. Gels were dried and scanned with a phosphorimager.

Labeled DNA or RNA oligonucleotide ligation assays were performed as described above but using 22.5 μM unlabeled CRISPR PCR fragment and ˜0.25 μM 5′ -end-labeled gel-purified oligonucleotides. Control assays were performed without adding CRISPR PCR fragment. For nuclease treatment of oligonucleotide ligation to CRISPR DNA, reactions were scaled up fourfold, treated with phenol-CIA, and ethanol-precipitated. The precipitated nucleic acids were dissolved in 30 μl of water. Equal amounts were then either untreated or treated with RNase H (2 units, Invitrogen), DNase I (RNase-free, 10 units, Roche), RNase A/T1mix [0.5 mg RNaseA (Sigma) and 500 units RNase T1 (Ambion)] in 40 mM tris (pH 7.9), 10 mM NaCl, 6 mM MgCl2, and 1 mM CaCl2 for 20 min at 37° C. Samples were extracted with phenol-CIA to terminate the reaction and analyzed by electrophoresis in a denaturing polyacrylamide gel, as described above. Labeled cDNA extension reactionswere carried out as above but using cold CRISPR DNA and oligonucleotides with 0.25 mM unlabeled dATP, dGTP, and dTTP and 5 mCi [α-³²P]-dCTP (3000 Ci/mMol, PerkinElmer). Oligonucleotides for cleavage/ligations assays were as follows: 29-nt DNA (TTTGGATCCTCATCTTTTAGGGCTCCAAG, SEQ ID NO: 38), 33-nt dsDNA-top (GATGCTTATGGTTATTGCAGCTACCCTCGCCCT, SEQ ID NO: 39), 33-nt dsDNA-bottom (AGGGCGAGGGTAGCTGCAATAACCATAAGCATC, SEQ ID NO: 40), 21-nt RNA (GCCGCUUCAGAGAGAAAUCGC, SEQ ID NO: 41), and 35-nt RNA (UUACGGUGCUUAAAACAAAACAAAACAAAACAAAA, SEQ ID NO: 42).

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Baltimore, D., RNA-dependent DNA polymerase in virions of RNA tumour viruses. Nature 226, 1209-1211, 1970.
Barrangou et al., CRISPR provides acquired resistance against viruses in prokaryotes. Science 315, 1709-1712, 2007.
Belfort et al., Genetic delineation of functional components of the group I intron in the phage T4 td gene. Cold Spring Harb. Symp. Quant. Biol. 52, 181-192, 1987.
Biebricher and Orgel, An RNA that multiplies indefinitely with DNA-dependent RNA polymerase: Selection from a random copolymer. Proc. Natl. Acad. Sci. U.S.A. 70, 934-938, 1973.
Blocker et al., Domain structure and three-dimensional model of a group II intron-encoded reverse transcriptase. RNA 11, 14-28, 2005.
Blumenthal and Carmichael, RNA replication: Function and structure of Qbeta-replicase. Annu. Rev. Biochem. 48, 525-548, 1979.
Boeke et al., Ty elements transpose through an RNA intermediate. Cell 40, 491-500 m 1985.
Bolotin et al., Clustered regularly interspaced short palindrome repeats (CRISPRs) have spacers of extrachromosomal origin. Microbiology 151, 2551-2561, 2005.
Brouns et al., Small CRISPR RNAs guide antiviral defense in prokaryotes. Science 321, 960-964, 2008.
Datsenko et al., Molecular memory of prior infections activates the CRISPR/Cas adaptive bacterial immunity system. Nat. Commun. 3, 945, 2012. doi: 10.1038/ncomms1937; pmid: 22781758
Flores et al., Viroids: Survivors from the RNA world? Annu. Rev. Microbiol. 68, 395-414, 2014.
Goldberg et al., Conditional tolerance of temperate phages via transcription-dependent CRISPR-Cas targeting. Nature 514, 633-637, 2014.
Greider and Blackburn, Identification of a specific telomere terminal transferase activity in tetrahymena extracts. Cell 43, 405-413, 1985.
Grynberg et al., DNA processing-related domain present in the anthrax virulence plasmid, pXO1. Trends Biochem. Sci. 29, 106-110, 2004.
Haas et al., How deep is deep enough for RNA-Seq profiling of bacterial transcriptomes? BMC Genomics 13, 734, 2012.
Hale et al., Essential features and rational design of CRISPR RNAs that function with the Cas RAMP module complex to cleave RNAs. Mol. Cell 45, 292-302, 2012.
Hale et al., RNA-guided RNA cleavage by a CRISPR RNACas protein complex. Cell 139, 945-956, 2009.
Heler et al., Cas9 specifies functional viral targets during CRISPR-Cas adaptation. Nature 519, 199-202, 2015.
Kim et al., Crystal structure of Cas1 from Archaeoglobus fulgidus and characterization of its nucleolytic activity. Biochem. Biophys. Res. Commun. 441, 720-725, 2013.
Konarska and Sharp, Replication of RNA by the DNA-dependent RNA polymerase of phage T7. Cell 57, 423-431, 1989.
Lambowitz and Zimmerly, Mobile group II introns. Annu. Rev. Genet. 38, 1-35 (2004). Lindner, et. al., 2008.
Liu et al., Reverse transcriptase-mediated tropism switching in Bordetella bacteriophage. Science 295, 2091-2094, 2002.
Ludwig and Klenk, Bergey's Manual of Systematic Bacteriology, 2:49-65, 2001.
Makarova et al., A putative RNA-interference-based immune system in prokaryotes: Computational analysis of the predicted enzymatic machinery, functional analogies with eukaryotic RNAi, and hypothetical mechanisms of action. Biol. Direct 1, 7, 2006.
Makarova et al., An updated evolutionary classification of CRISPR-Cas systems. Nat. Rev. Microbiol. 13, 722-736, 2015.
Makarova et al., Evolution and classification of the CRISPR-Cas systems. Nat. Rev. Microbiol. 9, 467-477, 2011.
Malik et al., The age and evolution of non-LTR retrotransposable elements. Mol. Biol. Evol. 16, 793-805, 1999.
Marraffini and Sontheimer, CRISPR interference limits horizontal gene transfer in staphylococci by targeting DNA. Science 322, 1843-1845, 2008.
Marraffini and Sontheimer, CRISPR interference: RNAdirected adaptive immunity in bacteria and archaea. Nat. Rev. Genet. 11, 181-190, 2010.
Mohr et al., Mechanisms used for genomic proliferation by thermophilic group II introns. PLOS Biol. 8, e1000391, 2010.
Mohr et al., Thermostable group II intron reverse transcriptase fusion proteins and their use in cDNA synthesis and next-generation RNA sequencing. RNA 19, 958-970, 2013.
Mojica et al., Intervening sequences of regularly spaced prokaryotic repeats derive from foreign genetic elements. J. Mol. Evol. 60, 174-182, 2005.
Moore and Sauer, The tmRNA system for translational surveillance and ribosome rescue. Annu. Rev. Biochem. 76, 101-124, 2007.
Nuñez et al., Integrase-mediated spacer acquisition during CRISPR-Cas adaptive immunity. Nature 519, 193-198, 2015.
Peng et al., She, An archaeal CRISPR type III-B system exhibiting distinctive RNA targeting features and mediating dual RNA and DNA interference. Nucleic Acids Res. 43, 406-417, 2015.
Pourcel et al., CRISPR elements in Yersinia pestis acquire new repeats by preferential uptake of bacteriophage DNA, and provide additional tools for evolutionary studies. Microbiology 151, 653-663, 2005.
Samai et al., Co-transcriptional DNA and RNA cleavage during Type III CRISPR-Cas immunity. Cell 161, 1164-1174, 2015.
Simon and Zimmerly, A diversity of uncharacterized reverse transcriptases in bacteria. Nucleic Acids Res. 36, 7219-7229, 2008.
Solano and Sanchez-Amat, Studies on the phylogenetic relationships of melanogenic marine bacteria: Proposal of Marinomonas mediterranea sp. nov. Int. J. Syst. Bacteriol. 49, 1241-1246, 1999.
Solano et al., Marinomonas mediterranea MMB-1 transposon mutagenesis:Isolation of a multipotent polyphenol oxidase mutant. J. Bacteriol. 182, 3754-3760 (2000).
Tamulaitis et al., Programmable RNA shredding by the type III-A CRISPR-Cas system of Streptococcus thermophilus. Mol. Cell 56, 506-517, 2014.
Temin and Mizutani, RNA-dependent DNA polymerase in virions of Rous sarcoma virus. Nature 226, 1211-1213, 1970.
Toro and Nisa-Martinez, Comprehensive phylogenetic analysis of bacterial reverse transcriptases. PLOS ONE 9, el14083, 2014.
van der Oost et al., E. R. Westra, R. N. Jackson, B. Wiedenheft, Unravelling the structural and mechanistic basis of CRISPRCas systems. Nat. Rev. Microbiol. 12, 479-492 , 2014.
Wei et al., Cas9 function and host genome sampling in Type II-A CRISPR-Cas adaptation. Genes Dev. 29, 356-361, 2015.
Xiong and Eickbush, Origin and evolution of retroelements based upon their reverse transcriptase sequences, 9, 3353-3362, 1990.
Yosef et al., Proteins and DNA elements essential for the CRISPR adaptation process in Escherichia coli. Nucleic Acids Res. 40, 5569-5576, 2012.
Zimmerly et al., Group II intron mobility occurs by target DNA-primed reverse transcription. Cell 82, 545-554, 1995.

1. A method for ligating RNA to DNA to provide a RNA-DNA hybrid comprising:
(a) obtaining RNA and a target DNA comprising a Cas1 recognition sequence; and
(b) providing a reverse transcriptase (RT) and a Cas1 protein, thereby producing a RNA-DNA hybrid.

2. The method of claim 1, wherein the RNA is ssRNA.

3. The method of claim 1, wherein the RT protein is at least 85% identical to SEQ ID NO: 6.

4. The method of claim 1, wherein the Cas1 protein is at least 85% identical to SEQ ID NO: 7.

5. The method of claim 1, wherein the RT and Cas1 protein are provided as a RT-Cas1 fusion protein.

6. The method of claim 5, wherein the RT-Cas1 fusion protein is a bacterial RT-Cas1 fusion protein.

7. The method of claim 6, wherein the RT-Cas1 fusion protein is from Arthrospira platensis or Marinomonas mediterranea.

8. The method of claim 1, wherein the RNA is 20-50 nucleotides.

9. The method of claim 1, wherein the RT and/or Cas1 protein is recombinant.

10. The method of claim 1, wherein the method is performed in the presence of added dNTPs.

11. The method of claim 1, wherein providing the RT and Cas1 protein comprises providing an expression vector that encodes the RT and Cas1 protein.

12. The method of claim 1, wherein step (b) further comprises providing a Cas2 polypeptide.

13. The method of claim 11, wherein the method is performed in a bacterial cell.

14. The method of claim 11, wherein the method is performed in a eukaryotic cell.

15. The method of claim 13, wherein the cell is comprised in an organism.

16. The method of claim 1, wherein the Cas1 recognition sequence comprises a CRISPR repeat sequence.

17. The method of claim 16, wherein the CRISPR repeat sequence comprises SEQ ID NO: 1 (GTTTCAGACCCGCTGGCCGCTTAGGCCGTTGAGAC).

18. A RNA-DNA hybrid produced according to the method of claim 1.

19-52. (canceled)

53. A isolated population of polynucleotides comprising a population of DNA-RNA chimeric molecules, each molecule comprising:
(i) a first dsDNA region;
(ii) a DNA/RNA region comprising one RNA strand and a complementary DNA strand; and
(iii) a second dsDNA region.

54-62. (canceled)

63. An expression construct comprising a sequence encoding (i) a RT and a Cas1 protein or a RT-Cas1 fusion protein; and (ii) comprising a sequence encoding a CRISPR adaptation gene.

64-82. (canceled)


SEQUENCE LISTING (42 sequences)

SEQ ID NO: 1 - DNA, 35 bp, Artificial sequence
  Synthetic oligonucleotide
gtttcagacccgctggccgcttaggccgttgagac

SEQ ID NO: 2 - DNA, 20 bp, Artificial sequence
  Synthetic oligonucleotide
ttggaaaaaataagggtact

SEQ ID NO: 3 - protein, 957 aa, Artificial sequence
  Synthetic polypeptide
MLNSPLIDAVLPLRSVVITLRWLSPSKTGFLHHAGLHAWVRFLAGSPEQFSDFIVVEPIENGHISYQAGDGYRFRITVLNGGESLLDTLFSSLKRLPESAANHPDIAGAFSDNLVLEKIEDTFEHHQVTQIEDLSVFDINALMLETAVWSRQRRFKVAFNTPARLVKPKPEDGTELKGQNRYCRDKSDLNWQLFTHRLTDTFINLFQSRTGERLQRQNWPEAQLHAGLAVWLNNSYTNKKEKKVKDASGMLAQMQIEIDDDFPADLLALLVLGQYIGMGQNRAFGMGQYQLQDAYGYCSYPRPQAAKSLLEKSLSDASLHQACQTMYPRQANFDSSDTDEEHHDAIDELLTKLYVSRERIFKREFTPSQLHSVEIEKPEGGTRLLSVPNWHDRTLQKAVTECLGNTLEHIWMKHSYGYRKGHSRLQARDQINQYIQQGYEWVLESDIESFFDSVNWLNLEQRLKLLLPNEPLVPLLMQWVSAAKQTEDEQTLARHNGLPQGAPISPILANLLLDDLDQDMIAKGHQIVRYADDFVLLFKSKAAAESALDDIITALKEHHLAINLEKTRIVEASQGFRYLGYLFVDGYAIETKREYRKEHAQLDKQLNASSLENEPSLQQEPAVQNEQSTLIGEREKLGTLLIIAGDIAMLSSEKQRLIVEQYDELHTYPWATLSSVLLVGPHHITTPALKSAMFHNVPVHFASQYGRYQGVSAGAAPSVFGADFWLLQAQYLQQETNALNISQVLIQARIEGIRAVISRREKDAPELNKIQRLDEKRLRAETLDQLRGYEGQASKQLWAFFQRILEEDWGFTGRNRRPPKDPINALLSLGYTYLYSLVDSVNRTVGLYPWQGALHQRHGYHHTLASDLMEPWRYLVEHVVLTLINRHQIHKDDFVIKENGCEMSSGARKTLLKELLVQLTKVPKGGNSLLTEMSNQSYRLALSCKMQQRFIAWSPKR

SEQ ID NO: 4 - protein, 90 aa, Artificial sequence
  Synthetic polypeptide
MRIYLACFDIEDDKKRRKLSNLLLEYGDRVQYSVFEISLKDENELHKLRKKCSKYTEEADSLRFYWLNKESRKHSQDVWGNPIAVFPAAV

SEQ ID NO: 5 - protein, 607 aa, Artificial sequence
  Synthetic polypeptide
TKLYVSRERIFKREFTPSQLHSVEIEKPEGGTRLLSVPNWHDRTLQKAVTECLGNTLEHIWMKHSYGYRKGHSRLQARDQINQYIQQGYEWVLESDIESFFDSVNWLNLEQRLKLLLPNEPLVPLLMQWVSAAKQTEDEQTLARHNGLPQGAPISPILANLLLDDLDQDMIAKGHQIVRYADDFVLLFKSKAAAESALDDIITALKEHHLAINLEKTRIVEASQGFRYLGYLFVDGYAIETKREYRKEHAQLDKQLNASSLENEPSLQQEPAVQNEQSTLIGEREKLGTLLIIAGDIAMLSSEKQRLIVEQYDELHTYPWATLSSVLLVGPHHITTPALKSAMFHNVPVHFASQYGRYQGVSAGAAPSVFGADFWLLQAQYLQQETNALNISQVLIQARIEGIRAVISRREKDAPELNKIQRLDEKRLRAETLDQLRGYEGQASKQLWAFFQRILEEDWGFTGRNRRPPKDPINALLSLGYTYLYSLVDSVNRTVGLYPWQGALHQRHGYHHTLASDLMEPWRYLVEHVVLTLINRHQIHKDDFVIKENGCEMSSGARKTLLKELLVQLTKVPKGGNSLLTEMSNQSYRLALSCKMQQRFIAWSPKR

SEQ ID NO: 6 - protein, 239 aa, Artificial sequence
  Synthetic polypeptide
TKLYVSRERIFKREFTPSQLHSVEIEKPEGGTRLLSVPNWHDRTLQKAVTECLGNTLEHIWMKHSYGYRKGHSRLQARDQINQYIQQGYEWVLESDIESFFDSVNWLNLEQRLKLLLPNEPLVPLLMQWVSAAKQTEDEQTLARHNGLPQGAPISPILANLLLDDLDQDMIAKGHQIVRYADDFVLLFKSKAAAESALDDIITALKEHHLAINLEKTRIVEASQGFRYLGYLFVDGYAI

SEQ ID NO: 7 - protein, 319 aa, Artificial sequence
  Synthetic polypeptide
TLLIIAGDIAMLSSEKQRLIVEQYDELHTYPWATLSSVLLVGPHHITTPALKSAMFHNVPVHFASQYGRYQGVSAGAAPSVFGADFWLLQAQYLQQETNALNISQVLIQARIEGIRAVISRREKDAPELNKIQRLDEKRLRAETLDQLRGYEGQASKQLWAFFQRILEEDWGFTGRNRRPPKDPINALLSLGYTYLYSLVDSVNRTVGLYPWQGALHQRHGYHHTLASDLMEPWRYLVEHVVLTLINRHQIHKDDFVIKENGCEMSSGARKTLLKELLVQLTKVPKGGNSLLTEMSNQSYRLALSCKMQQRFIAWSPKR

SEQ ID NO: 8 - DNA, 47 bp, Artificial sequence
  Synthetic oligonucleotide
attaccttaagcgtaaagacgcagcacgttacactagtttgatcaaa

SEQ ID NO: 9 - DNA, 47 bp, Artificial sequence
  Synthetic oligonucleotide
attaccttgggtctaaagacgcagcacgttacactagtttgatcaaa

SEQ ID NO: 10 - DNA, 57 bp, Artificial sequence
  Synthetic oligonucleotide
gagagtgatacgtgatctcgtaaatcccctcgtatcaattatatagtcgcaaacgac

SEQ ID NO: 11 - DNA, 57 bp, Artificial sequence
  Synthetic oligonucleotide
gagagtgatacgtgatctcttgggtctcctcgtatcaattatatagtcgcaaacgac

SEQ ID NO: 12 - DNA, 103 bp, Artificial sequence
  Synthetic oligonucleotide
gtttcagacccgctggccgcttaggccgttgagactaattgatacgaggagacccaagagatcacgtagtttcagacccgctggccgcttaggccgttgagac

SEQ ID NO: 13 - DNA, 85 bp, Artificial sequence
  Synthetic oligonucleotide
tagttttcgtcgtttgcgactatataattgatacgaggagacccaagagatcacgtatcactctcgacatgcacctcagggttcg

SEQ ID NO: 14 - DNA, 85 bp, Artificial sequence
  Synthetic oligonucleotide
tagttttcgtcgtttgcgactatataattgatacgaggggatttacgagatcacgtatcactctcgacatgcacctcagggttcg

SEQ ID NO: 15 - DNA, 46 bp, Artificial sequence
  Synthetic oligonucleotide
  misc_feature (18)..(22): n is a, c, g, or t
cgacgctcttccgatctnnnnnctgaaatgattggaaaaaataagg

SEQ ID NO: 16 - DNA, 36 bp, Artificial sequence
  Synthetic oligonucleotide
actgacgctagtgcatcacgtggcggagatctttaa

SEQ ID NO: 17 - DNA, 83 bp, Artificial sequence
  Synthetic oligonucleotide
  misc_feature (25)..(32): n is a, c, g, or t
caagcagaagacggcatacgagatnnnnnnnngtgactggagttcagacgtgtgctcttccgatcactgacgctagtgcatca

SEQ ID NO: 18 - DNA, 70 bp, Artificial sequence
  Synthetic oligonucleotide
  misc_feature (30)..(37): n is a, c, g, or t
aatgatacggcgaccaccgagatctacacnnnnnnnnacactctttccctacacgacgctcttccgatct

SEQ ID NO: 19 - DNA, 27 bp, Artificial sequence
  Synthetic oligonucleotide
  misc_feature (1)..(6): n is a, c, g, or t
nnnnnnagatcggaagagcacacgtct

SEQ ID NO: 20 - DNA, 60 bp, Artificial sequence
  Synthetic oligonucleotide
agatcggaagagcgtcgtgtcactcagtgactggagttcagacgtgtgctcttccgatct

SEQ ID NO: 21 - DNA, 58 bp, Artificial sequence
  Synthetic oligonucleotide
aatgatacggcgaccaccgagatctacactctttccctacacgacgctcttccgatct

SEQ ID NO: 22 - DNA, 60 bp, Artificial sequence
  Synthetic oligonucleotide
  misc_feature (25)..(30): n is a, c, g, or t
caagcagaagacggcatacgagatnnnnnngtgactggagttcagacgtgtgctcttccg

SEQ ID NO: 23 - DNA, 24 bp, Artificial sequence
  Synthetic oligonucleotide
cttagcgacgtagacctagttttt

SEQ ID NO: 24 - DNA, 24 bp, Artificial sequence
  Synthetic oligonucleotide
ggttattaagctgctaaagcgtag

SEQ ID NO: 25 - DNA, 38 bp, Artificial sequence
  Synthetic oligonucleotide
  misc_feature (18)..(22): n is a, c, g, or t
cgacgctcttccgatctnnnnngattcgcatggtaaac

SEQ ID NO: 26 - DNA, 36 bp, Artificial sequence
  Synthetic oligonucleotide
actgacgctagtgcatcaaactagtgtaacgtgctg

SEQ ID NO: 27 - DNA, 37 bp, Artificial sequence
  Synthetic oligonucleotide
  misc_feature (18)..(22): n is a, c, g, or t
cgacgctcttccgatctnnnnncacgaacctgaggtg

SEQ ID NO: 28 - DNA, 42 bp, Artificial sequence
  Synthetic oligonucleotide
actgacgctagtgcatcacgtcgtttgcgactatataattga

SEQ ID NO: 29 - DNA, 45 bp, Artificial sequence
  Synthetic oligonucleotide
  misc_feature (18)..(22): n is a, c, g, or t
cgacgctcttccgatctnnnnncacattcatgaccaccattctcg

SEQ ID NO: 30 - DNA, 40 bp, Artificial sequence
  Synthetic oligonucleotide
actgacgctagtgcatcacttcggtcttagcgacgtagac

SEQ ID NO: 31 - DNA, 42 bp, Artificial sequence
  Synthetic oligonucleotide
  misc_feature (18)..(22): n is a, c, g, or t
cgacgctcttccgatctnnnnnggggtgacatggtttcgacg

SEQ ID NO: 32 - DNA, 42 bp, Artificial sequence
  Synthetic oligonucleotide
actgacgctagtgcatcagcaggttattaagctgctaaagcg

SEQ ID NO: 33 - DNA, 180 bp, Artificial sequence
  Synthetic oligonucleotide
  misc_feature (65)..(76): n is a, c, g, or t
  misc_feature (147)..(158): n is a, c, g, or t
taaaaacattgaaggtctacaaggtcactttaaagctcacattcatgaccaccattctcgtcgcnnnnnnnnnnnnatggtaaaccaacgtcgtaagttgttggattaccagctgcgtaaagacgcagcacgttacactagtttgannnnnnnnnnnngtctacgtcgctaagaccgaag

SEQ ID NO: 34 - DNA, 180 bp, Artificial sequence
  Synthetic oligonucleotide
  misc_feature (21)..(32): n is a, c, g, or t
  misc_feature (101)..(112): n is a, c, g, or t
ggggtgacatggtttcgacgnnnnnnnnnnnncctgaggtgcatgtcgagagtgatacgtgatctcagctgtcccctcgtatcaattatatagtcgcaaannnnnnnnnnnncgctttagcagcttaataacctgctagtgtgctgccctcaggttgcttgtagcccgagattccgcagt

SEQ ID NO: 35 - DNA, 35 bp, Artificial sequence
  Synthetic oligonucleotide
ctgggacatataatatcgtccccgtagatgcctat

SEQ ID NO: 36 - DNA, 24 bp, Artificial sequence
  Synthetic oligonucleotide
cactcgaccggaattatcgacgaa

SEQ ID NO: 37 - DNA, 30 bp, Artificial sequence
  Synthetic oligonucleotide
tctgaaactctgaatactaacgaaaaatag

SEQ ID NO: 38 - DNA, 29 bp, Artificial sequence
  Synthetic oligonucleotide
tttggatcctcatcttttagggctccaag

SEQ ID NO: 39 - DNA, 33 bp, Artificial sequence
  Synthetic oligonucleotide
gatgcttatggttattgcagctaccctcgccct

SEQ ID NO: 40 - DNA, 33 bp, Artificial sequence
  Synthetic oligonucleotide
agggcgagggtagctgcaataaccataagcatc

SEQ ID NO: 41 - RNA, 21 nt, Artificial sequence
  Synthetic oligoribonucleotide
gccgcuucagagagaaaucgc

SEQ ID NO: 42 - RNA, 35 nt, Artificial sequence
  Synthetic oligoribonucleotide
uuacggugcuuaaaacaaaacaaaacaaaacaaaa